(19) United States
(12) Patent Application Publication
(10) Pub. No.: US 2003/0134883 A1
(43) Pub. Date: Jul. 17, 2003

(54) 14-Methyl-epothilones

(76) Inventors: David Myles, Kensington, CA (US); Kurt Sundermann, Burlingame, CA (US); Steven Dong, San Francisco, CA (US)

Correspondence Address: Gary Ashley, KOSAN Biosciences, Inc., 3832 Bay Center Place, Hayward, CA, 94545, US

(21) Appl. No.: 10/304,315

(22) Filed: Nov. 26, 2002

Related U.S. Application Data
(60) Provisional application No. 60/333,465, filed on Nov. 26, 2001.

(51) Int. Cl. A61K 31/427; A61K 31/423; C07D 417/02; C07D 413/02

(52) U.S. Cl. 514/365; 548/204; 548/236; 514/374

(57) ABSTRACT

The present invention provides 14-methyl epothilone compounds, along with intermediates thereto, methods for their preparation, compositions comprising the compounds, and methods for their use in the treatment of cancer and other diseases and conditions characterized by undesired cellular hyperproliferation.

### CROSS REFERENCE TO RELATED APPLICATIONS

This application claims priority under 35 U.S.C. §119(e) to U.S. provisional patent application serial No. 60/333,465, filed Nov. 26, 2001, which is incorporated herein by reference.

### BACKGROUND

The epothilones are natural products from the myxobacterium Sorangium cellulosum that possess potent antitumor activity due to their ability to stabilize microtubules. Several members of the epothilone family have been isolated:

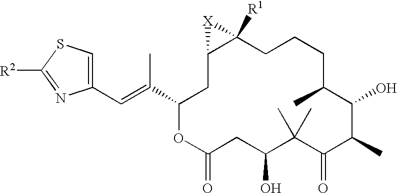

| | R¹ | R² | X |
|---|---|---|---|
| A | H | Me | O |
| B | Me | Me | O |
| C | H | Me | bond |
| D | Me | Me | bond |
| E | H | CH₂OH | O |
| F | Me | CH₂OH | O |

Epothilones A, B, E, and F are characterized by a 12,13-epoxide, which is replaced with a C═C double bond in epothilones C and D. Epothilones A, C, and E lack a 12-substituent, while epothilones B, D, and F have a 12-methyl group. Epothilones E and F have a hydroxylated methyl group on the thiazole. A number of minor epothilone analogs have been isolated. See Gerth et al., “Epothilons A and B: antifungal and cytotoxic compounds from Sorangium cellulosum (myxobacteria),” J. Antibiotics (1996) 49: 560-3; Hardt et al., “New natural epothilones from Sorangium cellulosum, strains So ce90/B2 and So ce90/D13: isolation, structure elucidation, and SAR studies,” J. Nat. Prod. (2001) 64: 847-856; and PCT publication WO 99/65913 (each of which is incorporated herein by reference).

Several total syntheses of epothilones have been reported. See Balog et al., “Total synthesis of (−)-epothilone A,” Angew. Chem. Int. Ed. Engl. (1996) 35: 2801-3; Meng et al., “Total syntheses of epothilones A and B,” J. Am. Chem. Soc. (1997) 119: 10073-92; Nicolaou et al., “Synthesis of epothilones A and B in solid and solution phase,” Nature (1997) 387: 268-272; Schinzer et al., “Total synthesis of (−)-epothilone A,” Angew. Chem. Int. Ed. Engl. (1997) 36: 523-4; Nicolaou et al., “Total synthesis of epothilone E and analogues with modified side chains through the Stille coupling reaction,” Angew. Chem. Int. Ed. Engl., (1998) 37: 84-87; Harris et al., “New chemical synthesis of the promising cancer therapeutic agent 12,13-desoxyepothilone B: discovery of a surprising long-range effect on the diastereoselectivity of an aldol condensation,” J. Am. Chem. Soc. (1999) 121: 7050-62; Schinzer et al., “Syntheses of (−)-epothilone B,” Chem. Eur. J. (1999) 5: 2492-99; Nicolaou et al., “Total synthesis of epothilone E and related side-chain modified analogues via a Stille coupling based strategy,” Bioorg Med Chem. (1999) 7:665-97; White et al., “A highly stereoselective synthesis of epothilone B,” J. Org. Chem. (1999) 64: 684-5; Lee et al., “Total synthesis and antitumor activity of 12,13-desoxyepothilone F: an unexpected solvolysis problem at C15, mediated by remote substitution at C21,” J. Org. Chem. (2000) 65: 6525-6533; Chappell et al., “Enroute to a plant scale synthesis of the promising antitumor agent 12,13-desoxyepothilone B,” Org. Lett. (2000) 2: 1633-6; Mulzer et al., “Total syntheses of epothilones B and D,” J. Org. Chem. (2000) 65: 7456-67; Sawada et al., “Enantioselective total synthesis of epothilones A and B using multifunctional asymmetric catalysis,” J. Am. Chem. Soc. (2000) 122: 10521-10532; Zhu & Panek, “Total synthesis of epothilone A,” Org. Lett. (2000) 2: 25775-2578; Taylor & Chen, “Total synthesis of epothilones B and D,” Org. Lett. (2001) 3: 2221-4; Bode & Carreira, “Stereoselective syntheses of apothilones A and B via directed nitrile oxide cycloaddition,” J. Am. Chem. Soc. (2001) 123: 3611-12; White et al., “Total synthesis of epothilone B, epothilone D, and cis- and trans-9,10-dehydroepothilone D,” J. Am. Chem. Soc. (2001) 123: 5407-5413; Martin & Thomas, “Total syntheses of epothilones B and D: applications of allylstannanes in organic synthesis,” Tet. Lett. (2001) 42:8373-8377, each of which is incorporated herein by reference. Preparation of epothilone fragments by degradation of epothilones has been disclosed in PCT publication WO 01/73103.

Several syntheses of epothilone analogs have been reported. See Su et al., “Structure-activity relationships of the epothilones and the first in vivo comparison with paclitaxel,” Angew. Chem. Int. Ed. Engl. (1997) 36: 2093-6; Borzilleri et al., “A novel application of a Pd(0)-catalyzed nucleophilic substitution reaction to the regio- and stereoselective synthesis of lactam analogues of the epothilone natural products,” J. Am. Chem. Soc. (2000) 122: 8890-7; Schinzer et al., “Synthesis and biological evaluation of aza-epothilones,” Chembiochem (2000) 1: 76-70; Altmann et al., “Synthesis and biological evaluation of highly potent analogues of epothilones B and D,” Bioorg. Med. Chem. Letts. (2000) 10: 2765-8; Johnson et al., “Synthesis, structure proof, and biological activity of spothilone cyclopropanes,” Org. Lett. (2000) 2: 1537-40; Nicolaou et al., “Total synthesis of 16-desmethylepothilone B, epothilone B10, epothilone F, and related side chain modified epothilone B analogues,” Chemistry (2000) 6:2783-800; Stachel et al., “On the interactivity of complex synthesis and tumor pharmacology in the drug discovery process: total synthesis and comparative in vivo evaluations of the 15-aza epothilones,” J. Org. Chem. (2001) 66: 4369-78; Nicolaou et al, “Synthesis and biological evaluation of 12,13-cyclopropyl and 12,13-cyclobutyl epothilones,” Chembiochem (2001) 1: 69-75; Nicolaou et al., “Chemical synthesis and biological evaluation of cis- and trans-12,13-cyclopropyl and 12,13-cyclobutyl epothilones and related pyridine side chain analogues,” J. Am. Chem. Soc. (2001) 123:9313-23, each of which is incorporated herein by reference.

A need exists for improved epothilones having improved activity, physical properties, and/or stability for use in the treatment of cancer and other diseases of cellular hyperproliferation. This invention addresses this and other needs by providing epothilone derivatives.

### SUMMARY OF THE INVENTION

In one aspect, the present invention provides compounds having the formula (I)

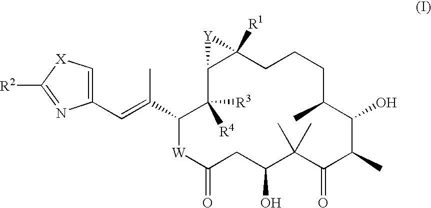

wherein R¹ is H or C₁-C₄ alkyl; R² is C₁-C₃ alkyl, CH₂OH, CH₂NH₂, or CH₂F; R³ is H and R⁴ is Me, or R³ is Me and R⁴ is H; W is O or NH; X is S or O; and Y is O or a bond. These compounds are useful in the treatment of diseases or conditions characterized by undesired cellular hyperproliferation.

In another aspect of the present invention compounds of the formula:

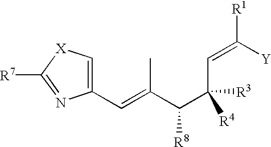

are provided wherein R¹ is H or C₁-C₄ alkyl; R³ is H and R⁴ is Me, or R³ is Me and R⁴ is H; R⁷ is C₁-C₃ alkyl, CH₂O(C═O)OCH₂CCl₃, CH₂N₃, CH₂NH(C═O)OCMe₃, or CH₂F; R⁸ is OH or NH₂; X is O or S; and Y is H, I, or CH═CH₂. These compounds are useful as intermediates in the preparation of compounds of formula (I).

In another aspect, the present invention provides methods for the preparation of compounds having the formula (I).

In another aspect, the present invention provides methods for the use of compounds having formula (I) in the treatment of diseases and conditions characterized by undesired cellular hyperproliferation.

### BRIEF DESCRIPTION OF THE FIGURES

FIG. 1 shows the therapeutic effect of (14S)-14-methylepothilone D against the MX-1 xenograft in nude mice as measured by a decrease in the rate of tumor growth after treatment.

FIG. 2 shows the effect of treatment with (14S)-14-methylepothilone D against the MX-1 exnograft in nude mice, measuring the body weight.

FIG. 3 shows a table of cytotoxicity against several cancer cell lines, measured in vitro.

### DETAILED DESCRIPTION OF THE INVENTION

Statements regarding the scope of the present invention and definitions of terms used herein are listed below. The definitions apply to the terms as they are used throughout this specification, unless otherwise limited in specific instances, either individually or as part of a larger group.

Some of the crystalline forms for the compounds may exist as polymorphs and as such are included in the present invention. In addition, some of the compounds may form solvates with water (i.e., hydrates) or common organic solvents, and such solvates are also encompassed within the scope of this invention.

Protected forms of the inventive compounds are included within the scope of the present invention. A variety of protecting groups are disclosed, for example, in T. H. Greene and P. G. M. Wuts, Protective Groups in Organic Synthesis, Third Edition, John Wiley & Sons, New York (1999), which is incorporated herein by reference in its entirety. For example, a hydroxy protected form of the inventive compounds are those where at least one of the hydroxyl groups is protected by a hydroxy protecting group. Illustrative hydroxy protecting groups include but not limited to tetrahydropyranyl; benzyl; methylthiomethyl; ethylthiomethyl; pivaloyl; phenylsulfonyl; triphenylmethyl; trisubstituted silyl such as trimethylsilyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl and the like; acyl and aroyl such as acetyl, pivaloylbenzoyl, 4-methoxybenzoyl, 4-nitrobenzoyl and aliphatic acylaryl and the like. Keto groups in the inventive compounds may similarly be protected.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in “Design of Prodrugs”, H. Bundgaard ed., Elsevier, 1985.

The term “subject” as used herein, refers to an animal, preferably a mammal, who has been the object of treatment, observation or experiment, and most preferably a human who has been the object of treatment and/or observation.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “composition” is intended to encompass a product comprising the specified ingredients in the specified amounts, as well as any product that results, directly or indirectly, from combinations of the specified ingredients in the specified amounts.

The term “pharmaceutically acceptable carrier” is a medium that is used to prepare a desired dosage form of the inventive compound. A pharmaceutically acceptable carrier includes solvents, diluents, or other liquid vehicle; dispersion or suspension aids; surface active agents; isotonic agents; thickening or emulsifying agents, preservatives; solid binders; lubricants and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe, ed. (Amer. Pharmaceutical Assoc. 2000), both of which are incorporated herein by reference in their entireties, disclose various carriers used in formulating pharmaceutical compositions and known techniques for the preparation thereof.

The term “pharmaceutically acceptable ester” is an ester that hydrolyzes in vivo into a compound of the present invention or a salt thereof. Illustrative examples of suitable ester groups include, for example, those derived from pharmaceutically acceptable aliphatic carboxylic acids such as formates, acetates, propionates, butyrates, acrylates, and ethylsuccinates.

In one aspect, the present invention provides compounds having the formula (I)

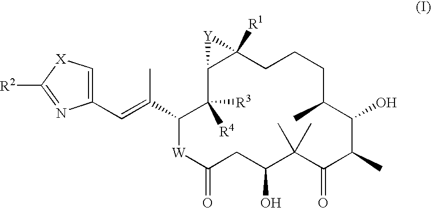

wherein R¹ is H or C1-C4 alkyl; R² is C₁-C₃ alkyl, CH₂OH, CH₂N₃, CH₂NH₂, or CH₂F; R³ is H and R⁴ is Me, or R³ is Me and R⁴ is H; W is O or NH; X is S or O; and Y is O or a bond.

One embodiment of the invention provides compounds of formula (II), compounds of formula (I) wherein Y is a bond:

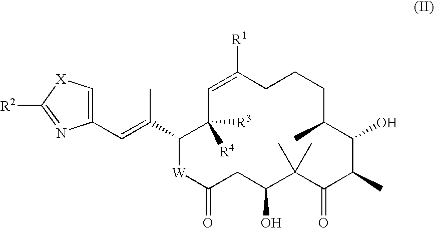

wherein R¹ is H or C1-C4 alkyl; R² is C₁-C₃ alkyl, CH₂OH, CH₂N₃, CH₂NH₂, or CH₂F; R³ is H and R⁴ is Me, or R³ is Me and R⁴ is H; W is O or NH; and X is S or O.

In one embodiment of the invention, compounds of formula (IIa) are provided

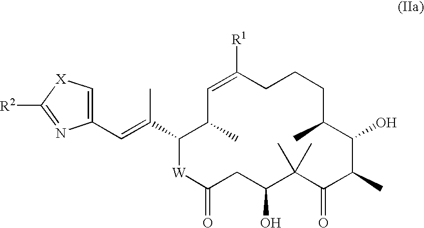

wherein R¹ is H or C1-C4 alkyl; R² is C₁-C₃ alkyl, CH₂OH, CH₂N₃, CH₂NH₂, or CH₂F; W is O or NH; and X is S or O.

In another embodiment of the invention, compounds of formula (IIa) are provided wherein R¹ is H or C1-C4 alkyl; R² is C₁-C₃ alkyl, CH₂OH, CH₂N₃, CH₂NH₂, or CH₂F; W is O or NH; and X is S.

In another embodiment of the invention, compounds of formula (IIa) are provided wherein R¹ is H or Me; R² is C₁-C₃ alkyl, CH₂OH, CH₂N₃, CH₂NH₂, or CH₂F; W is O or NH; and X is S.

In another embodiment of the invention, compounds of formula (IIa) are provided wherein R¹ is Me; R² is C₁-C₃ alkyl, CH₂OH, CH₂N₃, CH₂NH₂, or CH₂F; W is O or NH; and X is S.

In another embodiment of the invention, compounds of formula (IIa) are provided wherein R¹ is Me; R² is Me, CH₂OH, CH₂N₃, CH₂NH₂, or CH₂F; W is O or NH; and X is S.

In another embodiment of the invention, compounds of formula (IIa) are provided wherein R¹ is Me; R² is Me, CH₂OH, CH₂N₃, CH₂NH₂, or CH₂F; W is O; and X is S.

In another embodiment of the invention, compounds of formula (IIa) are provided wherein R¹ is Me; R² is Me, CH₂OH, CH₂N₃, CH₂NH₂, or CH₂F; W is NH; and X is S.

In another embodiment of the invention, compounds of formula (IIa) are provided having the structures

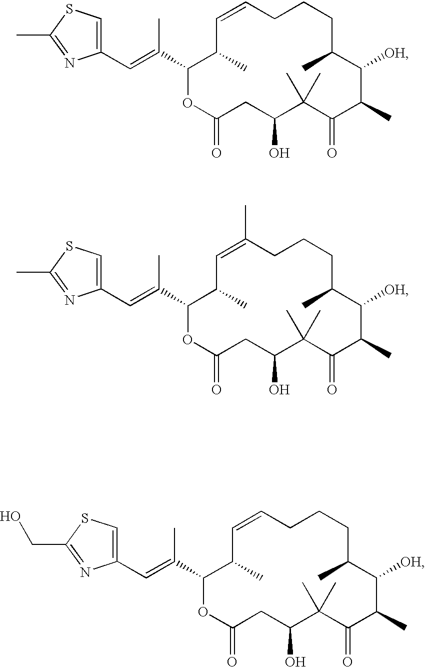

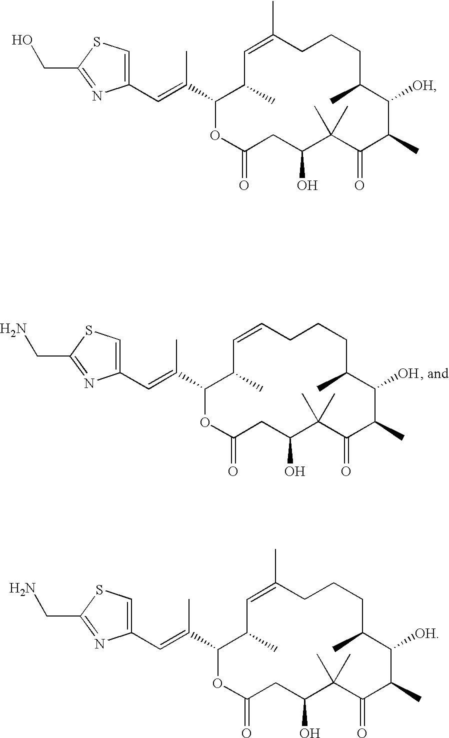

In another embodiment of the invention, compounds of formula (IIa) are provided having the structures

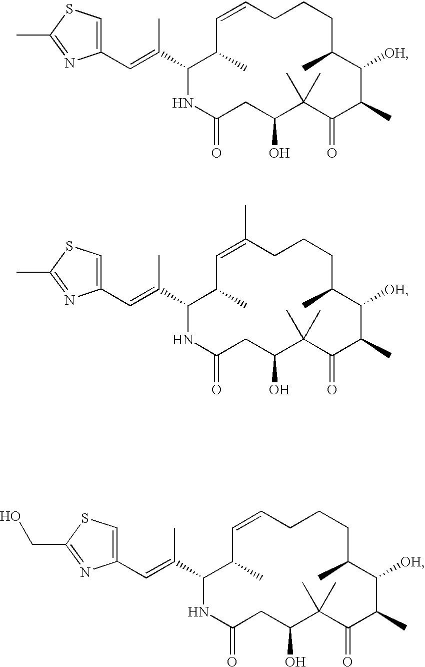

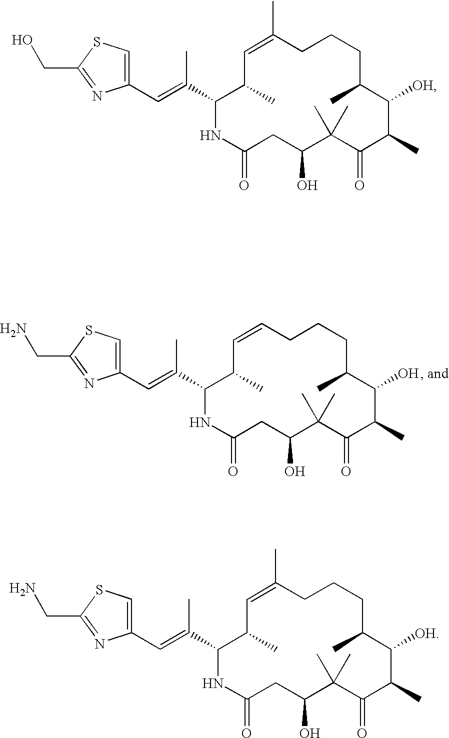

In another embodiment of the invention, compounds of formula (IIa) are provided wherein R¹ is H or C1-C4 alkyl; R² is C₁-C₃ alkyl, CH₂OH, CH₂N₃, CH₂NH₂, or CH₂F; W is O or NH; and X is O.

In another embodiment of the invention, compounds of formula (IIa) are provided wherein R¹ is H or Me; R² is C₁-C₃ alkyl, CH₂OH, CH₂N₃, CH₂NH₂, or CH₂F; W is O or NH; and X is O.

In another embodiment of the invention, compounds of formula (IIa) are provided wherein R¹ is Me; R² is C₁-C₃ alkyl, CH₂OH, CH₂N₃, CH₂NH₂, or CH₂F; W is O or NH; and X is O.

In another embodiment of the invention, compounds of formula (IIa) are provided wherein R¹ is Me; R² is Me, CH₂OH, CH₂N₃, CH₂NH₂, or CH₂F; W is O or NH; and X is O.

In another embodiment of the invention, compounds of formula (IIa) are provided wherein R¹ is Me; R² is Me, CH₂OH, CH₂N₃, CH₂NH₂, or CH₂F; W is O; and X is O.

In another embodiment of the invention, compounds of formula (IIa) are provided wherein R¹ is Me; R² is Me, CH₂OH, CH₂N₃, CH₂NH₂, or CH₂F; W is NH; and X is O.

In another embodiment of the invention, compounds of formula (IIa) are provided having the structures

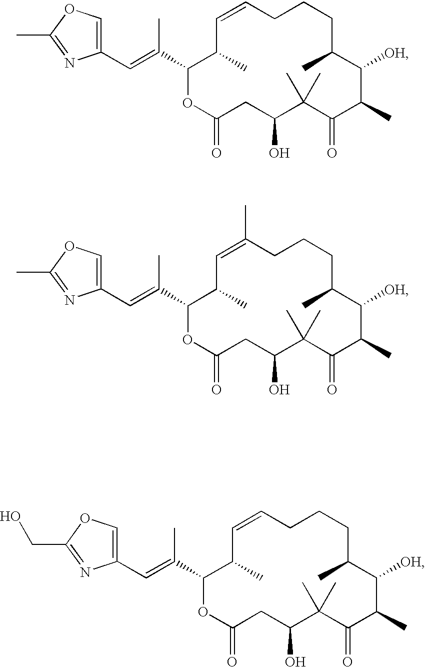

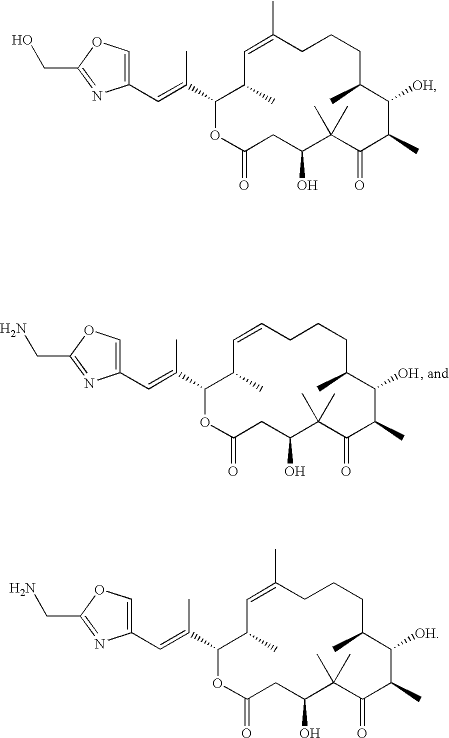

In another embodiment of the invention, compounds of formula (IIa) are provided having the structures

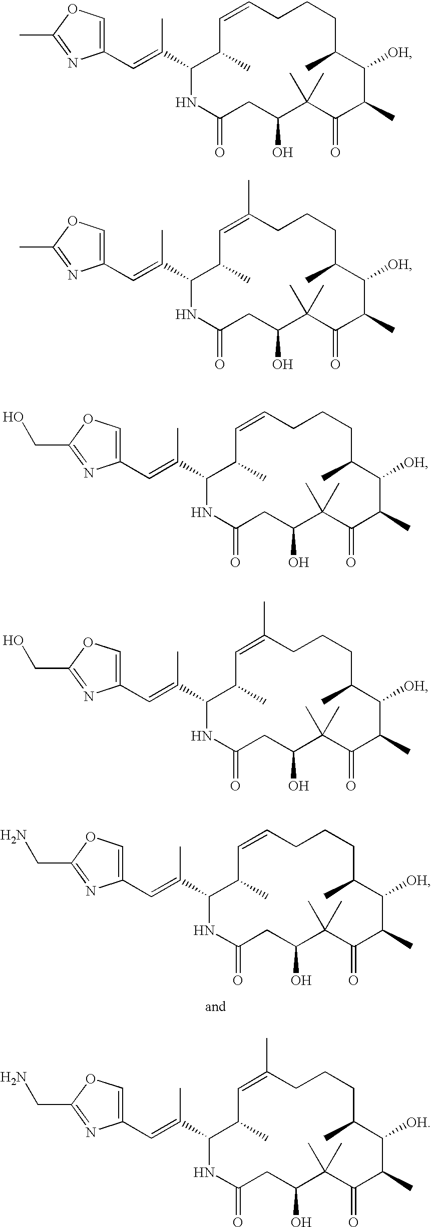

In another embodiment of the invention, compounds of formula (III), which are compounds of formula (I) wherein Y is O, are provided:

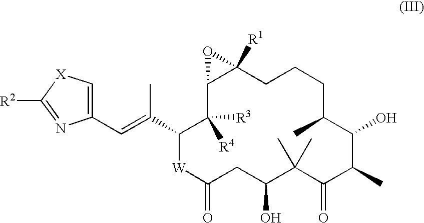

wherein R¹ is H or C1-C4 alkyl; R² is C₁-C₃ alkyl, CH₂OH, CH₂N₃, CH₂NH₂, or CH₂F; R³ is H and R⁴ is Me, or R³ is Me and R⁴ is H; W is O or NH; and X is S or O.

In one embodiment of the invention, compounds of formula (IIIa) are provided

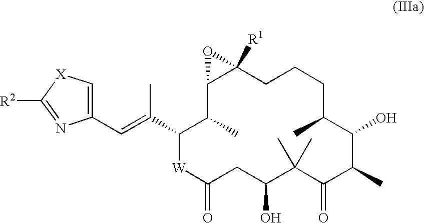

wherein R¹ is H or C1-C4 alkyl; R² is C₁-C₃ alkyl, CH₂OH, CH₂N₃, CH₂NH₂, or CH₂F; W is O or NH; and X is S or O.

In another embodiment of the invention, compounds of formula (IIIa) are provided wherein R¹ is H or C1-C4 alkyl; R² is C₁-C₃ alkyl, CH₂OH, CH₂N₃, CH₂NH₂, or CH₂F; W is O or NH; and X is S.

In another embodiment of the invention, compounds of formula (IIIa) are provided wherein R¹ is H or Me; R² is C₁-C₃ alkyl, CH₂OH, CH₂N₃, CH₂NH₂, or CH₂F; W is O or NH; and X is S.

In another embodiment of the invention, compounds of formula (IIIa) are provided wherein R¹ is Me; R² is C₁-C₃ alkyl, CH₂OH, CH₂N₃, CH₂NH₂, or CH₂F; W is O or NH; and X is S.

In another embodiment of the invention, compounds of formula (IIIa) are provided wherein R¹ is Me; R² is Me, CH₂OH, CH₂N₃, CH₂NH₂, or CH₂F; W is O or NH; and X is S.

In another embodiment of the invention, compounds of formula (IIIa) are provided wherein R¹ is Me; R² is Me, CH₂OH, CH₂N₃, CH₂NH₂, or CH₂F; W is O; and X is S.

In another embodiment of the invention, compounds of formula (IIIa) are provided wherein R¹ is Me; R² is Me, CH₂OH, CH₂N₃, CH₂NH₂, or CH₂F; W is NH; and X is S.

In another embodiment of the invention, compounds of formula (IIIa) are provided having the structures

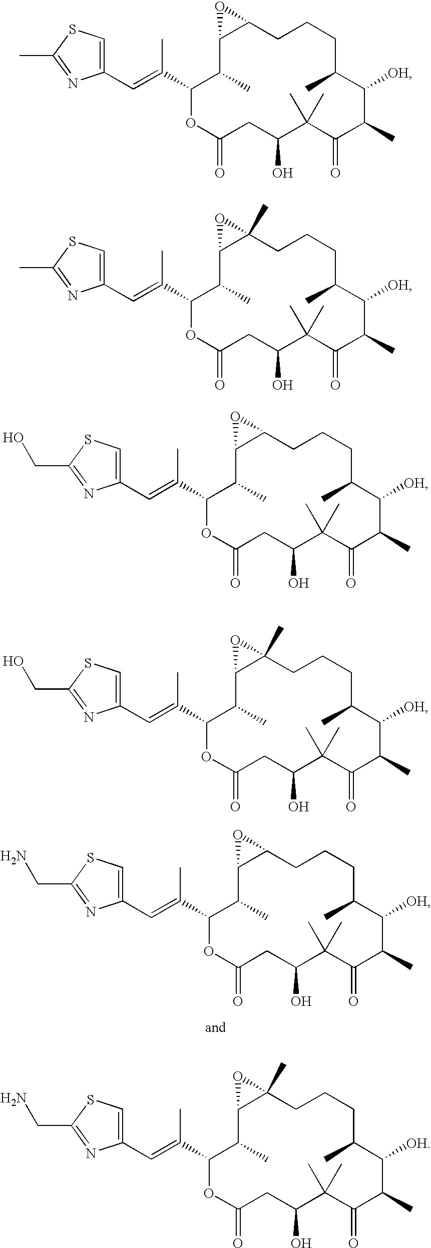

In another embodiment of the invention, compounds of formula (IIIa) are provided having the structures

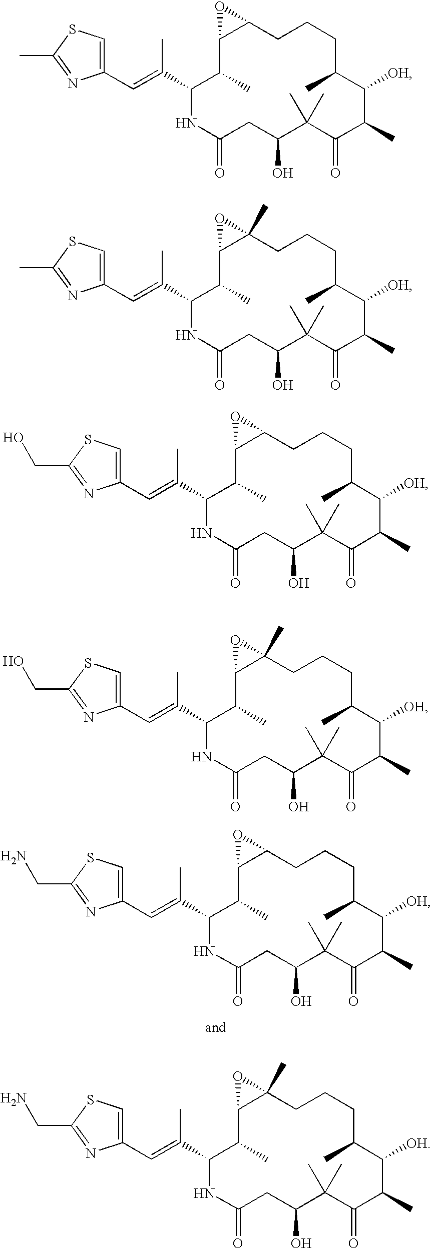

In another embodiment of the invention, compounds of formula (IIIa) are provided wherein R¹ is H or C1-C4 alkyl; R² is C₁-C₃ alkyl, CH₂OH, CH₂N₃, CH₂NH₂, or CH₂F; W is O or NH; and X is O.

In another embodiment of the invention, compounds of formula (IIIa) are provided wherein R¹ is H or Me; R² is C₁-C₃ alkyl, CH₂OH, CH₂N₃, CH₂NH₂, or CH₂F; W is O or NH; and X is O.

In another embodiment of the invention, compounds of formula (IIIa) are provided wherein R¹ is Me; R² is C₁-C₃ alkyl, CH₂OH, CH₂N₃, CH₂NH₂, or CH₂F; W is O or NH; and X is O.

In another embodiment of the invention, compounds of formula (IIIa) are provided wherein R¹ is Me; R² is Me, CH₂OH, CH₂N₃, CH₂NH₂, or CH₂F; W is O or NH; and X is O.

In another embodiment of the invention, compounds of formula (IIIa) are provided wherein R¹ is Me; R² is Me, CH₂OH, CH₂N₃, CH₂NH₂, or CH₂F; W is O; and X is O.

In another embodiment of the invention, compounds of formula (IIIa) are provided wherein R¹ is Me; R² is Me, CH₂OH, CH₂N₃, CH₂NH₂, or CH₂F; W is NH; and X is O.

In another embodiment of the invention, compounds of formula (IIIa) are provided having the structures

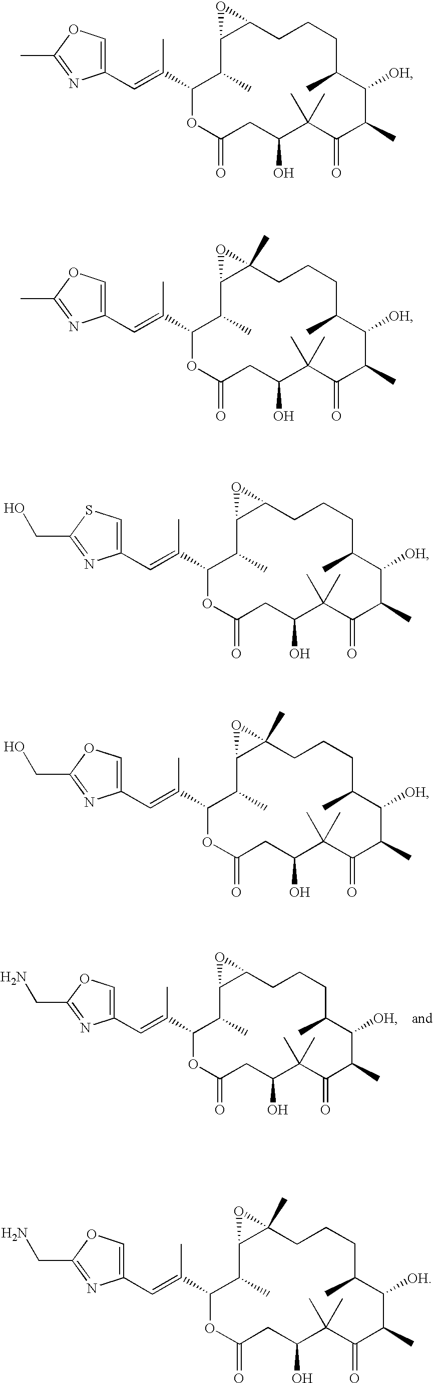

In another embodiment of the invention, compounds of formula (IIIa) are provided having the structures

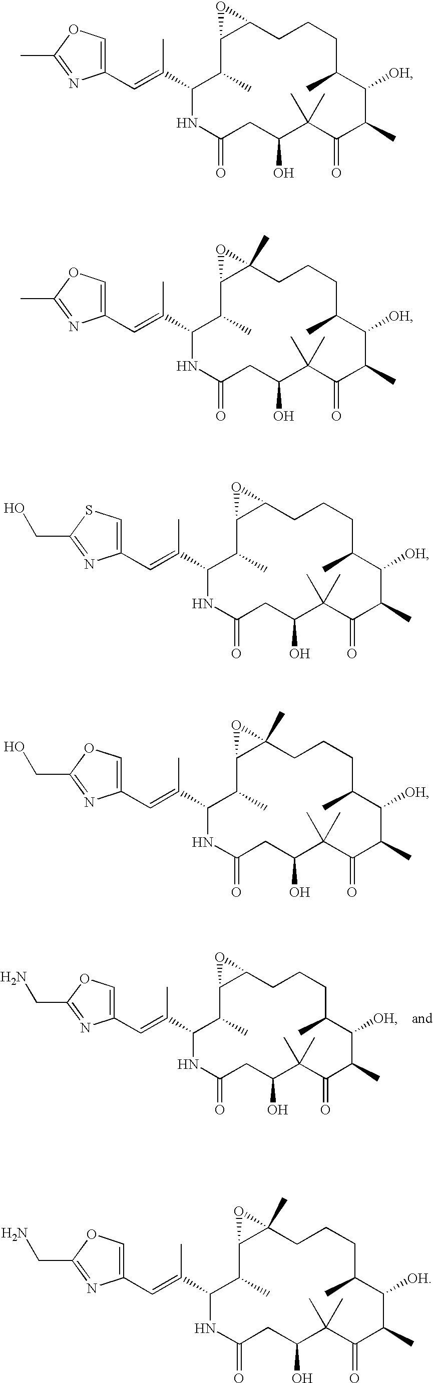

In another aspect of the present invention compounds of formula (IV) are provided:

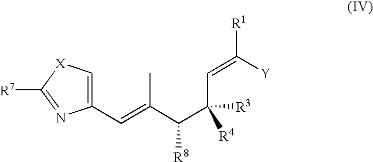

wherein R¹ is H or C₁-C₄ alkyl; R³ is H and R⁴ is Me, or R³ is Me and R⁴ is H; R⁷ is C₁-C₃ alkyl, CH₂O(C═O)OCH₂CCl₃, CH₂N₃, CH₂NH(C═O)OCMe₃, or CH₂F; R⁸ is OH or NH₂; X is O or S; and Y is I or CH═CH₂.

In one embodiment of the invention, compounds of formula (IVa) are provided

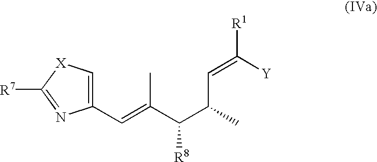

wherein R¹ is H or C₁-C₄ alkyl; R⁷ is C₁-C₃ alkyl, CH₂O(C═O)OCH₂CCl₃, CH₂N₃, CH₂NH(C═O)OCMe₃, or CH₂F; R⁸ is OH or NH₂; X is O or S; and Y is I or CH═CH₂.

In one embodiment of the invention, compounds of formula (IVa) are provided wherein R¹ is H or C₁-C₄ alkyl; R⁷ is C₁-C₃ alkyl, CH₂O(C═O)OCH₂CCl₃, CH₂N₃, CH₂NH(C═O)OCMe₃, or CH₂F; R⁸ is OH or NH₂; X is O or S; and Y is I.

In one embodiment of the invention, compounds of formula (IVa) are provided wherein R¹ is H or C₁-C₄ alkyl; R⁷ is C₁-C₃ alkyl, CH₂O(C═O)OCH₂CCl₃, CH₂N₃, CH₂NH(C═O)OCMe₃, or CH₂F; R⁸ is OH or NH₂; X is S; and Y is I.

In one embodiment of the invention, compounds of formula (IVa) are provided wherein R¹ is H or C₁-C₄ alkyl; R⁷ is C₁-C₃ alkyl, CH₂O(C═O)OCH₂CCl₃, CH₂N₃, CH₂NH(C═O)OCMe₃, or CH₂F; R⁸ is OH or NH₂; X is O; and Y is I.

In one embodiment of the invention, compounds of formula (IVa) are provided wherein R¹ is H or C₁-C₄ alkyl; R⁷ is C₁-C₃ alkyl, CH₂O(C═O)OCH₂CCl₃, CH₂N₃, CH₂NH(C═O)OCMe₃, or CH₂F; R⁸ is OH or NH₂; X is O or S; and Y is CH═CH₂.

In one embodiment of the invention, compounds of formula (IVa) are provided wherein R¹ is H or C₁-C₄ alkyl; R⁷is C₁-C₃ alkyl, CH₂O(C═O)OCH₂CCl₃, CH₂N₃, CH₂NH(C═O)OCMe₃, or CH₂F; R⁸ is OH or NH₂; X is S; and Y is CH═CH₂.

In another aspect of the present invention compounds of formula (V) are provided:

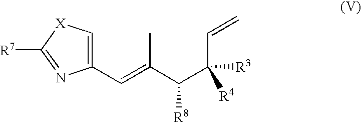

wherein R³ is H and R⁴ is Me, or R³ is Me and R⁴ is H; R⁷ is C₁-C₃ alkyl, CH₂O(C═O)OCH₂CCl₃, CH₂N₃, CH₂NH(C═O)OCMe₃, or CH₂F; R⁸ is OH or NH₂; and X is O or S.

In one embodiment of the invention, compounds of formula (Va) are provided

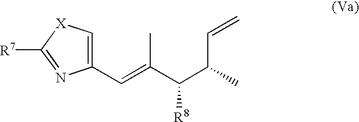

wherein R⁷ is C₁-C₃ alkyl, CH₂O(C═O)OCH₂CCl₃, CH₂N₃, CH₂NH(C═O)OCMe₃, or CH₂F; R⁸ is OH or NH₂; and X is O or S.

In one embodiment of the invention, compounds of formula (Va) are provided wherein R⁷ is C₁-C₃ alkyl, CH₂O(C═O)OCH₂CCl₃, CH₂N₃, CH₂NH(C═O)OCMe₃, or CH₂F; R⁸ is OH or NH₂; and X is O.

In one embodiment of the invention, compounds of formula (Va) are provided wherein R⁷ is C₁-C₃ alkyl, CH₂O(C═O)OCH₂CCl₃, CH₂N₃, CH₂NH(C═O)OCMe₃, or CH₂F; R⁸ is OH; and X is O.

In one embodiment of the invention, compounds of formula (Va) are provided wherein R⁷ is C₁-C₃ alkyl CH₂O(C═O)OCH₂CCl₃, CH₂N₃, CH₂NH(C═O)OCMe₃, or CH₂F; R⁸ is NH₂; and X is O.

In one embodiment of the invention, compounds of formula (Va) are provided wherein R⁷ is C₁-C₃ alkyl, CH₂O(C═O)OCH₂CCl₃, CH₂N₃, CH₂NH(C═O)OCMe₃, or CH₂F; R⁸ is OH or NH₂; and X is S.

In one embodiment of the invention, compounds of formula (Va) are provided wherein R⁷ is C₁-C₃ alkyl, CH₂O(C═O)OCH₂CCl₃, CH₂N₃, CH₂NH(C═O)OCMe₃, or CH₂F; R⁸ is OH; and X is S.

In one embodiment of the invention, compounds of formula (Va) are provided wherein R⁷ is C₁-C₃ alkyl, CH₂O(C═O)OCH₂CCl₃, CH₂N₃, CH₂NH(C═O)OCMe₃, or CH₂F; R⁸ is NH₂; and X is S.

In another aspect of the invention, methods are provided for the preparation of compounds of formula (I). In one embodiment of the invention, compounds of formula (IV) wherein Y is CH═CH₂ are used to prepare compounds of formula (I) as illustrated in Scheme 1 and Examples 10-15 and 30-31 below.

In this embodiment, reaction of a compound of formula (IV) wherein R¹ is H or C₁-C₄ alkyl; R³ is H and R⁴ is Me, or R³ is Me and R⁴ is H; R⁷ is C₁-C₃ alkyl, CH₂O(C═O)OCH₂CCl₃, CH₂N₃, CH₂NH(C═O)OCMe₃, or CH₂F; R⁸ is OH; X is O or S; and Y is CH═CH₂ with the fragment (1), wherein P¹ is a protecting group such as triethylsilyl or tert-butyldimethylsilyl and P² is a protecting group such as tert-butyldimethylsilyl or 2,2,2-trichloroethoxycarbonyl (Troc), in the presence of a condensing agent, for example 1-[3-(dimethylamino)propyl]-3-ethylcarbodiimide and 4-(dimethylamino)pyridine, provides ester (2), wherein W═O. Similarly, reaction of a compound of formula (IV) wherein R¹ is H or C₁-C₄ alkyl; R³ is H and R⁴ is Me, or R³ is Me and R⁴ is H; R⁷ is C₁-C₃ alkyl, CH₂O(C═O)OCH₂CCl₃, CH₂N₃, CH₂NH(C═O)OCMe₃, or CH₂F; R⁸ is NH₂; X is O or S; and Y is CH═CH₂ with the fragment (1), wherein P¹ is a protecting group such as trialkylsilyl, particularly triethylsilyl or tert-butyldimethylsilyl, and P² is a protecting group such as tert-butyldimethylsilyl or 2,2,2-trichloroethoxycarbonyl (Troc), in the presence of a condensing agent, for example 1-[3-(dimethylamino)propyl]-3-ethylcarbodiimide and 4-(dimethylamino)pyridine, provides amide (2), wherein W═NH.

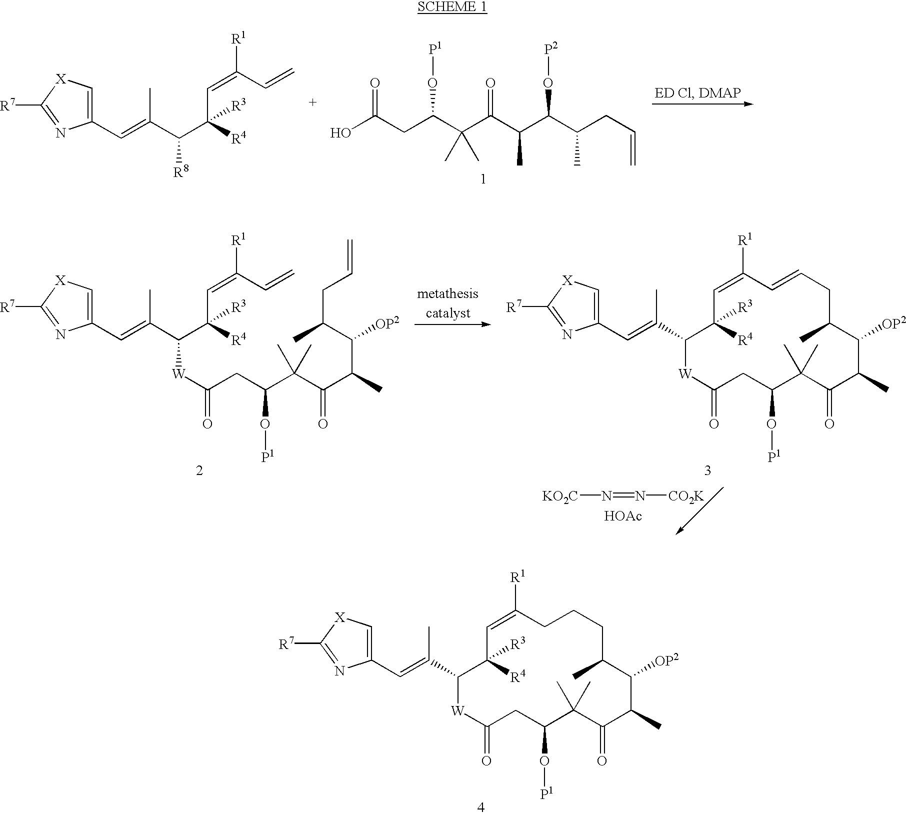

Macrocyclization to provide (3) is performed by treatment of (2) with a suitable olefin metathesis catalyst. Typical metathesis catalysts are complexes of ruthenium or molybdenum. In a preferred embodiment, the metathesis catalyst is tricyclohexyl-phosphine-[1,3-bis(2,4,6-trimethylphenyl)-4,5-dihydroimidazol-2-ylidene][benzylidene]-ruthenium(IV)dichloride. The 10,11-alkene is selectively reduced using diimide, generated from dipotassium azodicarboxylate in the presence of acetic acid, to provide the protected macrocycle (4).

Compound (4) is deprotected by removal of the Troc protecting groups using zinc metal and acetic acid or using samarium iodide, followed by removal of the trialkylsilyl groups and (C═O)OCMe₃ groups using either 80% HF/pyridine or trifluoroacetic acid (Scheme 2) to provide the compound of formula (I).

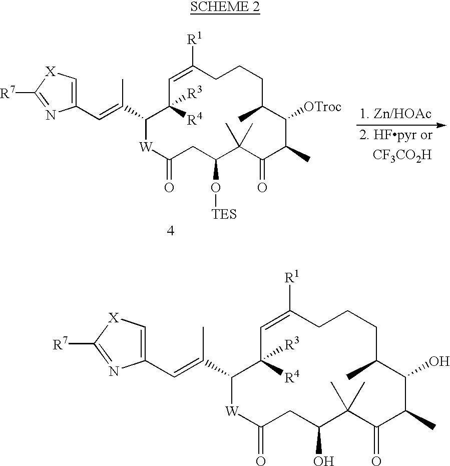

Compounds wherein R² is CH₂N₃ are converted into compounds wherein R² is CH₂NH₂ by treatment with trimethylphosphine in a mixture of tetrahydrofuran and water.

Fragment (1) wherein P¹ is triethylsilyl and P² is Troc can be prepared as illustrated in Scheme 3 starting from fragment (5), the preparation of which is described in Lee et al., “Insights into long-range structural effects on the stereochemistry of aldol condensations: a practical total synthesis of desoxyepothilone F,” J. Am. Chem. Soc. (2001) 123: 5249-5259, incorporated herein by reference.

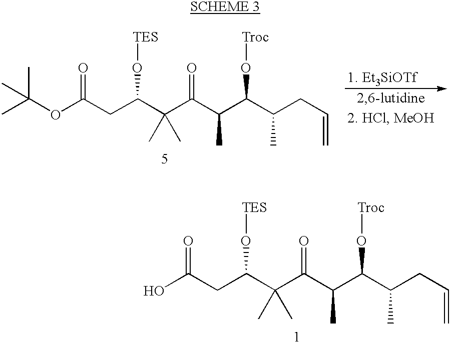

In another embodiment of the invention, compounds of formula (I) are prepared by joining compounds of formula (V) with a compound of formula (6) as illustrated in Scheme 4 and Examples 40-43 below.

In this embodiment, a compound of formula (V) wherein R³ is H and R⁴ is Me, or R³ is Me and R⁴ is H; R⁷ is C₁-C₃ alkyl, CH₂O(C═O)OCH₂CCl₃, CH₂N₃, CH₂NH(C═O)OCMe₃, or CH₂F; R⁸ is OH; and X is O or S is condensed with compound (6), wherein R¹ is H or C₁-C₄ alkyl and P is a hydroxyl protecting group such as a trialkylsilyl group, to produce the ester (7) wherein W is O. Similarly, condensation of a compound of formula (IV) wherein R³ is H and R⁴ is Me, or R³ is Me and R⁴ is H; R⁷ is C₁-C₃ alkyl, CH₂O(C═O)OCH₂CCl₃, CH₂N₃, CH₂NH(C═O)OCMe₃, or CH₂F; R⁸ is NH₂; X is O or S; and Y is H is condensed with compound (6), wherein R¹ is H or C₁-C₄ alkyl and P is a hydroxyl protecting group such as a trialkylsilyl group, to produce the amide (7) wherein W is NH. Suitable condensing agents include, for example, a carbodiimide such as 1-[3-(dimethyl-amino)-propyl]-3-ethylcarbodiimide or dicyclohexylcarbodiimide, together with 4-(dimethylamino)-pyridine. Compounds (7) is subjected to ring-forming olefin metathesis using a suitable metal catalyst, for example the Grubbs catalyst tricyclohexylphosphine-[1,3-bis(2,4,6-trimethylphenyl)-4,5-dihydroimidazol-2-ylidene][benzylidene]-ruthenium(IV)dichloride, to provide the protected macrocycle (8). Deprotection as described above provides the compound of formula (I). Details of this embodiment are presented in Example 43 below.

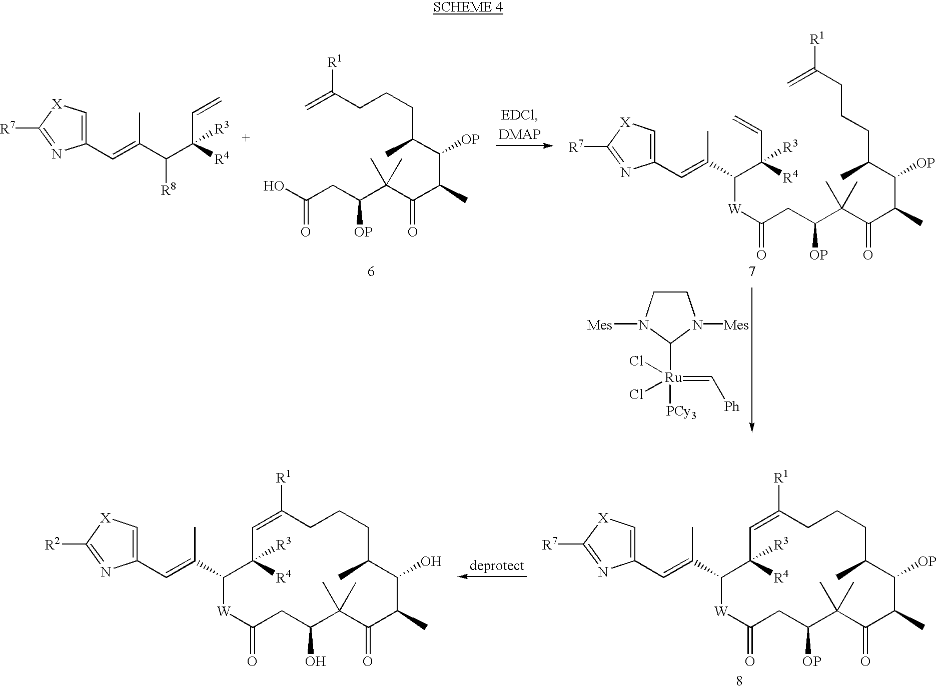

In one embodiment of the invention, compound (6) is prepared by degradation of an epothilone as illustrated in Scheme 5.

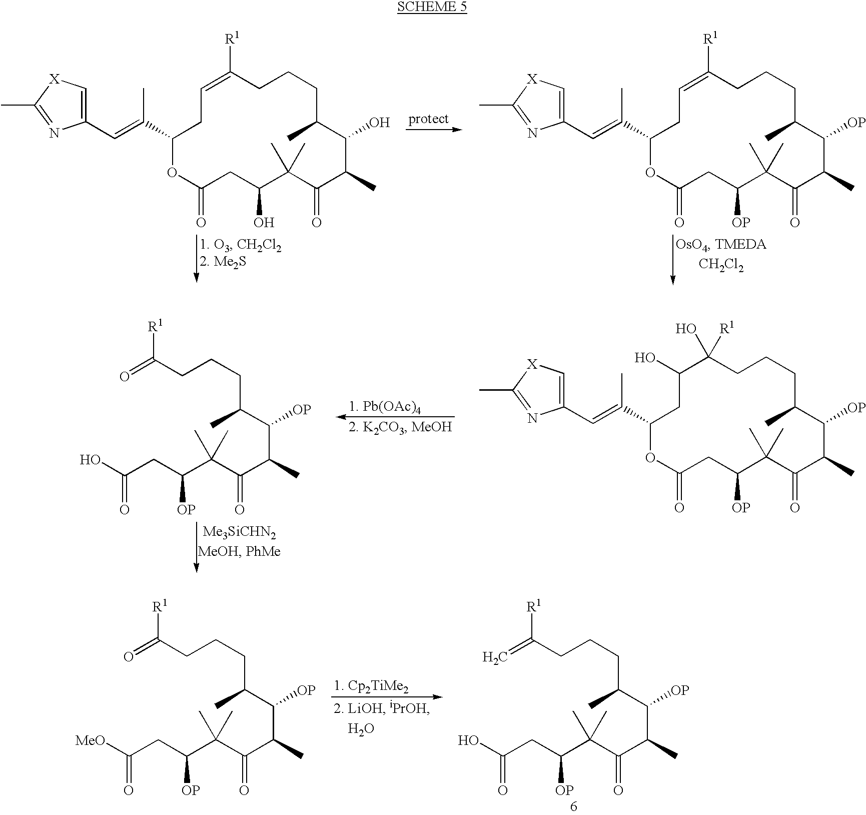

The epothilone is first protected at the 3- and 7-OH groups, for example as the trialkylsilyl ethers. In a preferred embodiment, P is tert-butyldimethylsilyl, as illustrated in Example 40. The 12,13-alkene is then cleaved using a two-step process wherein the alkene is first converted into the 12,13-diol by reaction with osmium tetraoxide and tetramethyl-ethylenediamine as described in Examples 41 and 42 below. The 12,13-diol is then cleaved by reaction with lead tetraacetate in benzene, followed by reaction with alkaline methanol to produce the ketoacid. Alternatively, the protected epothilone is subjected to ozonolysis with a reductive workup to provide the ketoacid in a one-step procedure. The ketoacid is next converted to the methyl ester, for example using diazomethane or (trimethylsilyl)-diazomethane, the ketone is converted to the alkene by reaction with dimethyltitanocene, and the methyl ester is hydrolyzed to provide (6).

In another embodiment of the invention, compounds of formula (III) are prepared from compounds of formula (I) by treatment with an epoxidizing agent as illustrated in Scheme 6. In a preferred embodiment, the epoxidizing agent is dimethyldioxirane. Details of this embodiment are provided below in Examples 32 and 33 below.

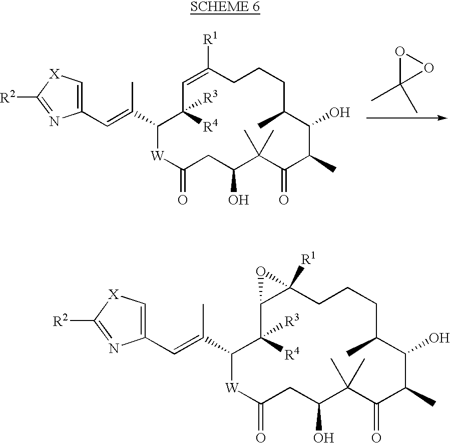

In another aspect of the present invention, methods are provided for the preparation of compounds of formula (IV) and (V). In one embodiment, illustrated in Scheme 7 and Examples 34-39, methods are provided for the preparation of compounds of formula (IVa) wherein R⁷ is R⁹, wherein R⁹ is C₁-C₃ alkyl or COOEt, and R⁸ is OH.

In this embodiment, an anti-selective aldol condensation between the aldehyde (9) and N-propionyl (4S)-4-benzyl-2-oxazolidinone provides the aldol adduct (10). The alcohol group of (10) is protected, for example as a trialkylsilyl ether using (R¹⁰)₃SiCl in the presence of imidazole in dimethylformamide or (R¹⁰)₃SiOTf in the presence of 2,6-lutidine in dichloromethane. In certain embodiments, (R¹⁰)₃SiCl is triethylsilyl chloride or tert-butyldimethylsilyl chloride. The chiral auxiliary is removed by reduction with lithium borohydride, and the resulting alcohol is oxidized to provide the aldehyde (13), for example using Swern conditions (oxalyl chloride, methylsulfoxide, and triethylamine), Corey-Kim conditions (N-chlorosuccinimide, methylsulfide, and diisopropylethylamine), or Pfizer-Moffat conditions (a carbodimide such as 1-[3-(dimethylamino)propyl]-3-ethylcarbodiimide with methylsulfoxide and pyridinium trifluoroacetate). The aldehyde (13) is reacted with a phosphorus ylid Ph₃P═C(R¹)Z, wherein Z is H or I, to provide the compound of formula (IVa). When the phosphorus ylid is Ph₃P═C(R¹)H, the compound of formula (IVa) wherein Y is H is obtained. When the phosphorus ylid is Ph₃P═C(R¹)I, the compound of formula (IVa) wherein Y is I is obtained.

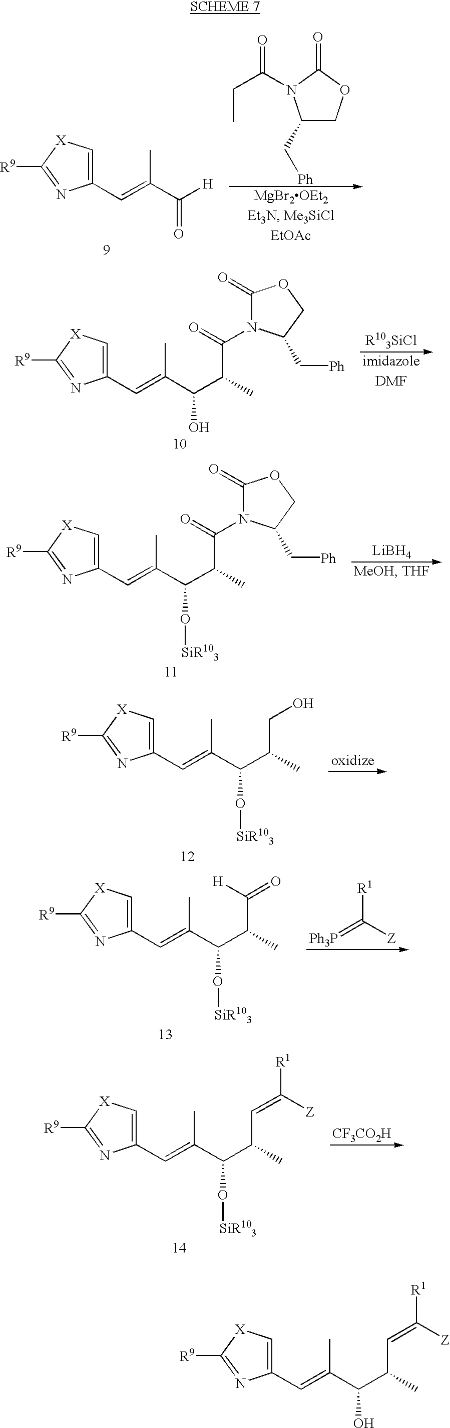

Scheme 8 illustrates one embodiment for making the phosphorus ylids. An alkyltriphenylphosphonium iodide is treated with butyllithium to form the ylid wherein Z is H. This ylid can be further converted into the ylid wherein Z is I by treatment with iodine to form the iodoalkyl triphenylphosphonium iodide, followed by treatment with sodium hexamethyldisilazide (NaHMDS) to produces a solution of the iodinated phosphorus ylid. The ylid wherein R¹ is H and Z is I may also be prepared by reaction of diiodomethane with triphenylphosphine to produce iodomethyltriphenylphosphonium iodide, followed by reaction with NaHMDS.

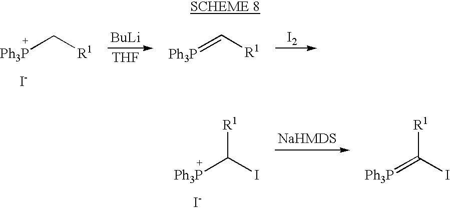

In another embodiment of the invention, compounds of formula (Va) wherein R⁷ is R⁹, wherein R⁹ is C₁-C₃ alkyl or COOEt, are prepared according to the method illustrated in Scheme 9.

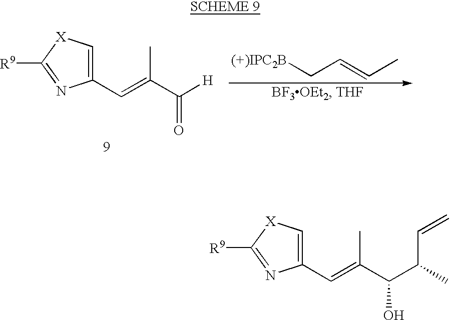

Reaction of the aldehyde (9) with the (+)-enantiomer of crotyl-diisopinocampheyl-borane in the presence of a Lewis acid, for example boron trifluoride etherate, directly yields the compound of formula (Va) wherein R⁷ is R⁹, wherein R⁹ is C₁-C₃ alkyl or COOEt.

The aldehydes (9) may be prepared as illustrated in Scheme 10.

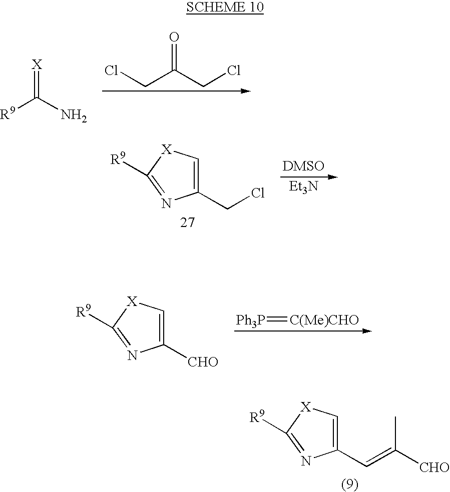

Reaction of an acetamide or thioacetamide R⁹(C═X)NH₂, wherein R⁹ is C₁-C₃ alkyl or COOEt and X is O or S with 1,3-dichloroacetone provides the chloromethyl oxazole or thiazole (27), respectively, which is oxidized by treatment with methylsulfoxide followed by triethylamine to provide the aromatic aldehyde. Treatment of the aromatic aldehyde with 2-(triphenylphosphoranylidene)-propionaldehyde provides compound (9).

In one embodiment, illustrated in Scheme 11 and Examples 18-27 below, methods are provided for the preparation of compounds of formula (IV) wherein R³ is H and R⁴ is Me; R⁷ is R⁹, wherein R⁹ is C₁-C₃ alkyl or COOEt, and R⁸ is OH.

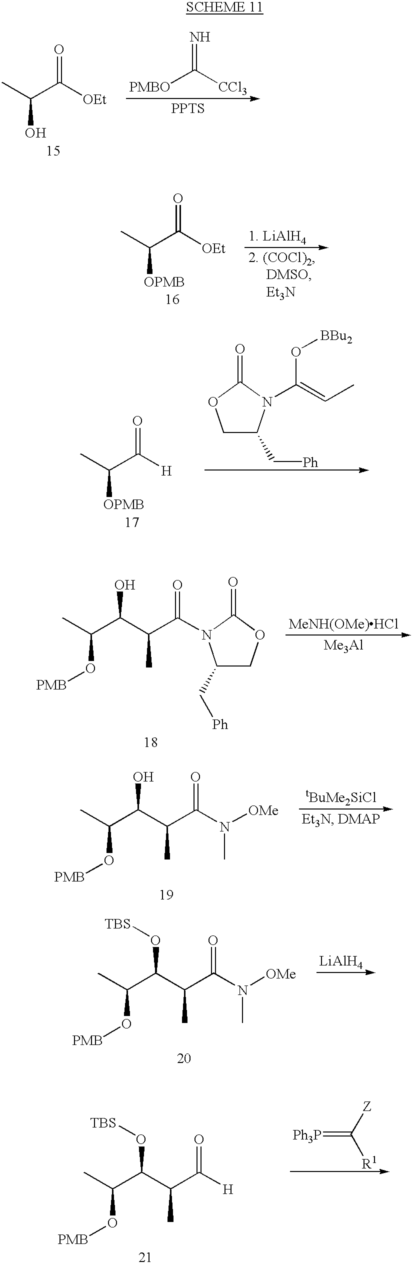

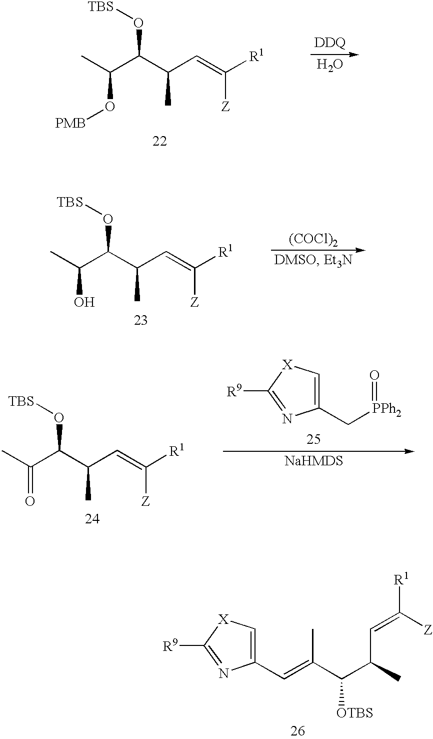

In this embodiment, the hydroxyl group of ethyl (S)-lactate (15) is protected by reaction with 4-methoxybenzyl trichloroacetimidate in the presence of an acid catalyst. Preferred examples of acid catalysts include pyridinium p-toluene-sulfonate (PPTS) and trifluoromethanesulfonic acid. The ester group of (16) is reduced to aldehyde (17), methods for which include using a reductant such as diisobutylaluminum hydride (DiBAlH) at −78° C., or using a two-step process wherein the ester is first reduced to an alcohol, for example using lithium aluminum hydride, followed by oxidation of the alcohol to the aldehyde, for example using Swern conditions (oxalyl chloride, methylsulfoxide, and triethylamine), Corey-Kim conditions (N-chlorosuccinimide, methylsulfide, and diisopropylethylamine), or Pfizer-Moffat conditions (a carbodiimide such as 1-[3-(dimethylamino)propyl]-3-ethylcarbodiimide with methylsulfoxide and pyridinium trifluoroacetate). The resulting PMB-protected (S)-lactaldehyde (3) is used in an Evans aldol condensation with the dibutylboron-enolate of (4R)-3-propionyl-4-benzyl-2-oxazolidinone to provide aldol adduct (18). Displacement of the oxazolidinone by reaction with N,O-dimethylhydroxylamine hydrochloride and trimethylaluminum provides Weinreb amide (19). Protection of the alcohol provides (20). In a preferred embodiment, this protection is achieved by treating (19) with tert-butyldimethylsilyl chloride, 4-(dimethylamino)pyridine, and triethylamine. In another embodiment, with tert-butyldimethylsilyl triflate and 2,6-lutidine are used to produce (20). Reduction of the Weinreb amide, for example using lithium aluminum hydride or DiBAlH, provides aldehyde (21), which is converted into the alkene (22) using the phosphorus ylid Ph₃P═C(R¹)Z as described above. The alkene (22) is deprotected using 2,3-dichloro-5,6-dicyano-1,4-benzoquinone (DDQ) in the presence of water to give alcohol (23). Oxidation to the ketone (24) is accomplished using, for example, Swern conditions (oxalyl chloride, methylsulfoxide, and triethylamine), Corey-Kim conditions (N-chlorosuccinimide, methylsulfide, and diisopropylethylamine), or Pfizer-Moffat conditions (a carbodiimide such as 1-[3-(dimethylamino)propyl]-3-ethylcarbodiimide, methylsulfoxide, and pyridinium trifluoroacetate). Ketone (24) is reacted with the ylid formed from phosphine oxide (25) and a strong base such as NaHMDS to provide compound (26). The compounds of formula (IVb) are obtained by deprotection of (26) using trifluoroacetic acid. The phosphine oxides (25) can be prepared by reaction of 2-chloromethyl heterocycle (27) described in Scheme 10 above with diphenylphosphine oxide in the presence of a base such as cesium carbonate.

In another embodiment of the invention, compounds of formula (IVa) are prepared as illustrated in Scheme 12 and Examples 1-7 below.

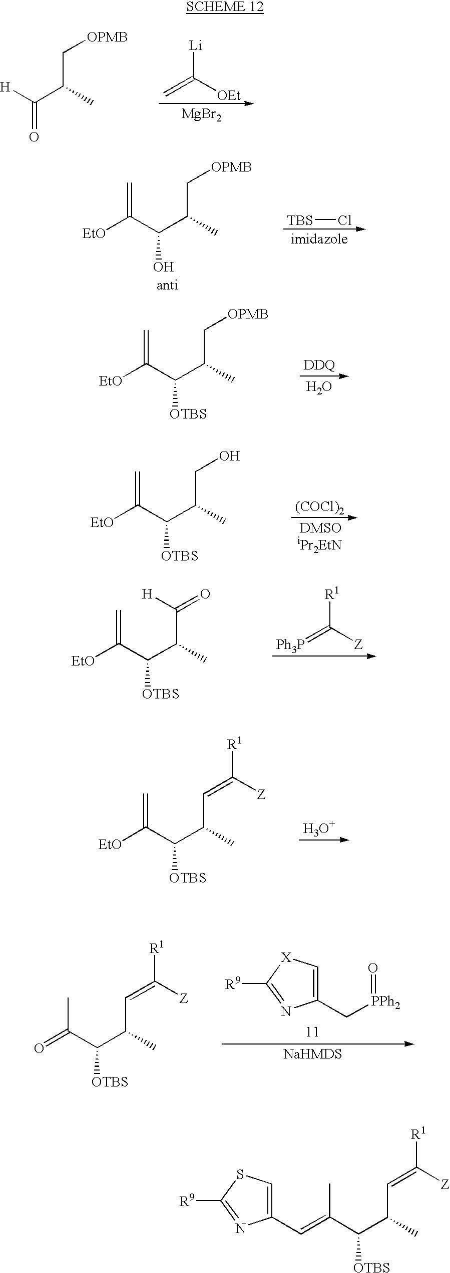

Reaction of lithiated ethyl vinyl ether with magnesium bromide followed by (S)-3-(4-methoxybenzyloxy)-2-methylpropanal, prepared according to Smith et al., “Gram-scale synthesis of (+)-discodermolide,” J. Am. Chem. Soc. (2000) 122: 8654-8664, which is incorporated herein by reference, provides the anti-addition product via chelation control. The alcohol is protected, prior to removal of the PMB ether. In a preferred embodiment, the alcohol is protected as a silyl ether, for example tert-butyldimethylsilyl. The PMB ether is cleaved by oxidation with DDQ in the presence of water, and the resulting alcohol is oxidized to the aldehyde, for example using Swern conditions (oxalyl chloride, methylsulfoxide, and triethylamine), Corey-Kim conditions (N-chlorosuccinimide, methylsulfide, and diisopropylethylamine), or Pfizer-Moffat conditions (a carbodiimide such as 1-[3-(dimethylamino)propyl]-3-ethylcarbodiimide, methylsulfoxide, and pyridinium trifluoroacetate). Addition of a phosphorus ylid as described above yields the vinyl ether. The vinyl ether is hydrolyzed using mild acid in water, and the resulting ketone is reacted with the thiazolylmethyl- or oxazolylmethyl-phosphine oxide as discussed above to yield the compounds of formula (IVa) in protected form. The tert-butyldimethylsilyl ether is cleaved, for example by treatment with trifluoroacetic acid or tetrabutylammonium fluoride, to provide the compound of formula (IVa).

In another embodiment of the invention, lithiated ethyl vinyl ether is added to (S)-3-(4-methoxybenzyloxy)-2-methylpropanal in the absence of magnesium bromide to provide both the syn- and anti-diastereomers of the alcohol product. These diastereomers are separated by chromatography, and the anti-product is used as described above in Scheme 12. The syn-diastereomer is used to prepare the corresponding epimer, which is used to prepare compounds of formula (IVa) wherein R⁸ is NH₂ as illustrated in Scheme 13.

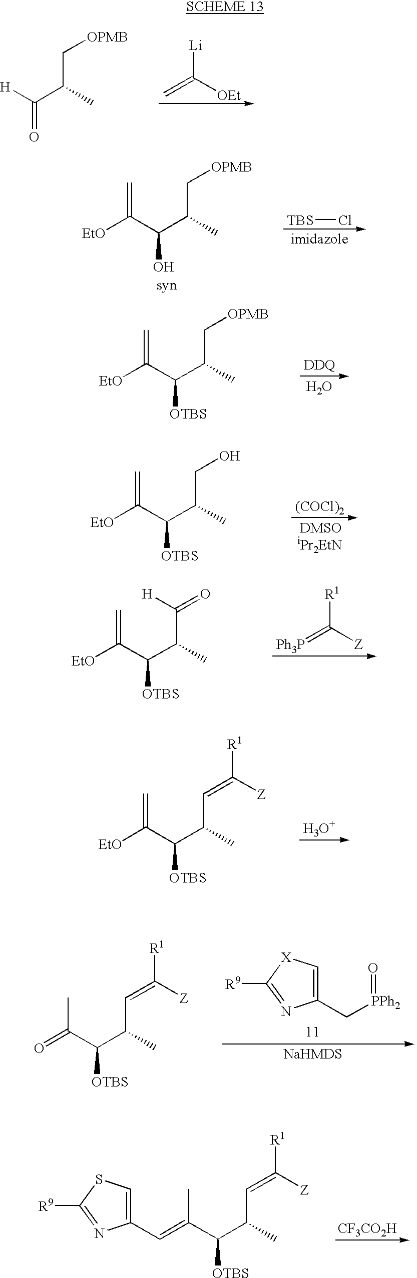

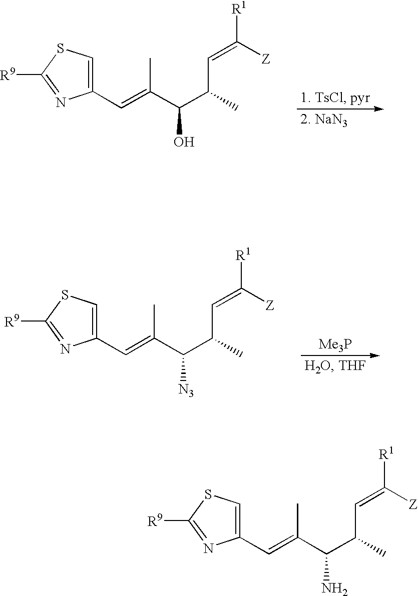

In another embodiment of the invention, compounds of formula (II) are prepared in a non-stereoselective manner by starting with the racemic aldehyde of Scheme 6. The addition of lithiated ethyl vinyl ether gives rise to racemic pairs of syn and anti diastereomeric alcohols, which is separated and carried forward. Once coupled to further chiral fragments discussed below, the racemic pairs of each diastereomeric alcohol is separated.

In another embodiment of the invention, compounds of formula (IV) wherein R⁷ is CH₂O(C═O)OCH₂CCl₃, CH₂N₃, CH₂NH(C═O)OCMe₃, or CH₂F are prepared from compound (26), as illustrated in Scheme 14.

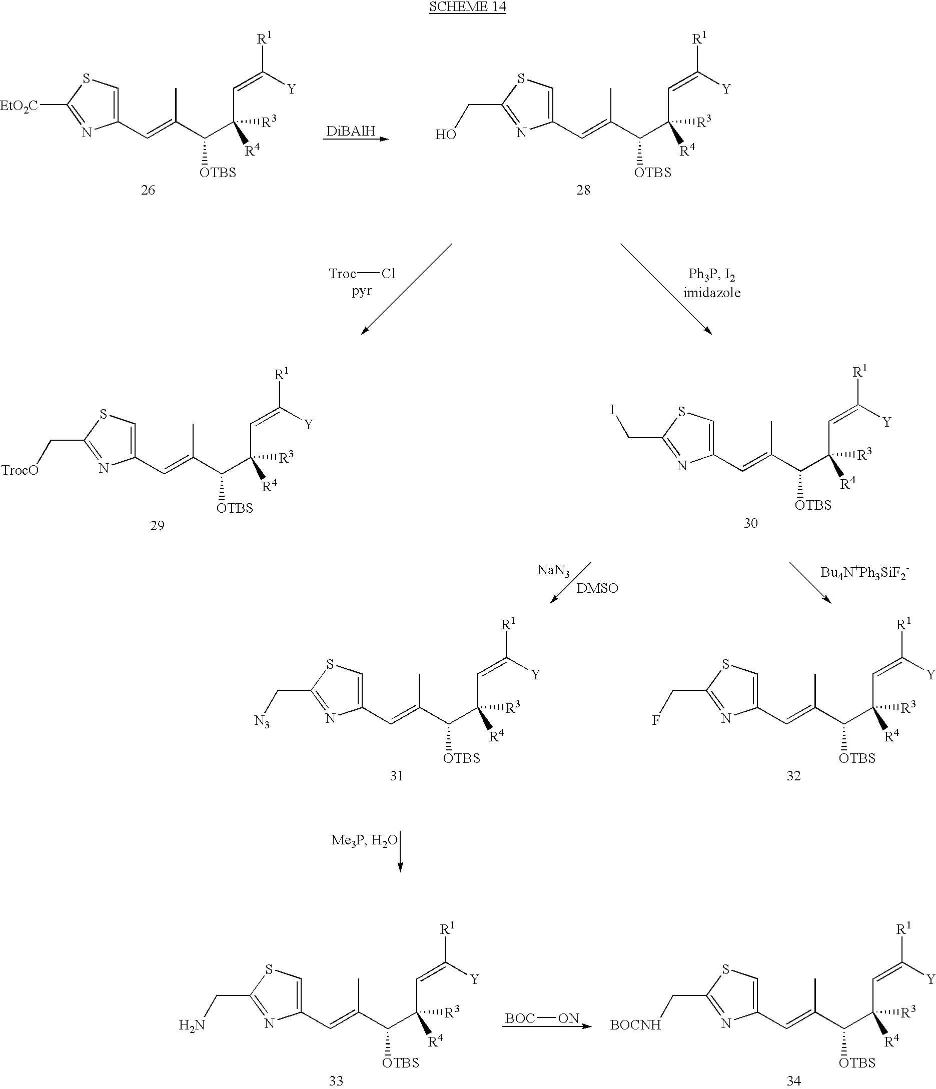

Reduction of the ester of compound (26) with diisobutylaluminum hydride provides (28). Treatment of (28) with 2,2,2-trichloroethyl chloroformate and pyridine provides the Troc-protected compound (29), which can be deprotected using trifluoroacetic acid to provide the compound of formula (IV) wherein R⁷ is CH₂O(C═O)OCH₂CCl₃. Treatment of (28) with iodine and triphenylphosphine in the presence of imidazole provides the iodide (30), which can be reacted with a source of nucleophilic fluorine, for example KF, tetrabutylammonium fluoride, or tetrabutylammonium triphenyldifluorosilicate, to provide (32). Compound (32) can be deprotected by treatment with trifluoroacetic acid to provide the compound of formula (IV) wherein R⁷ is CH₂F. Iodide (30) can be treated with sodium azide in methylsulfoxide to provide the azide (31), which can be deprotected by treatment with trifluoroacetic acid to provide the compound of formula (IV) wherein R⁷ is CH₂N₃. Azide (31) can be reduced by reaction with trimethylphosphine in aqueous tetrahydrofuran to provide the amine (33), which can be converted to the tert-butyl carbamate by reaction with di(tert-butyl)dicarbonate or BOC-ON, 2-(tert-butoxycarbonyloxyimino)-2-phenylacetonitrile, to provide (34). Compound (34) can be deprotected by treatment with tetrabutylammonium fluoride to provide the compound of formula (IV) wherein R⁷ is CH₂O(C═O)OCMe₃.

In another embodiment of the invention, the compounds of formula (IV) wherein Y is I are converted to compounds of formula (IV) wherein Y is CH═CH₂. As shown in Scheme 15 and detailed in Examples 8, 9, 28, and 29 below, reaction of the compounds of formula (IV) wherein Y is I with tributylvinylstannane, vinylboronic acid, or trimethoxyvinylsilane in the presence of a palladium catalyst provides compounds of formula (II) wherein Y is CH═CH₂. In a preferred embodiment, the reaction uses tributylvinylstannane and tetrakis(triphenylphosphine)-palladium.

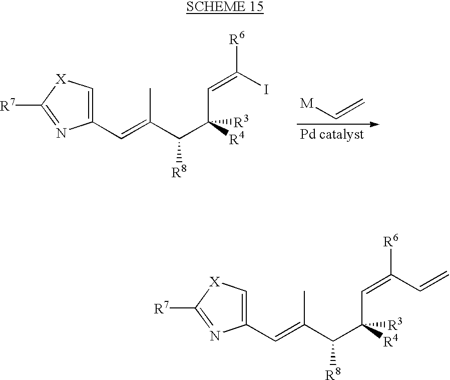

Formulation

A composition of the present invention generally comprises a compound of the present invention and a pharmaceutically acceptable carrier. The inventive compound may be in free form or where appropriate as pharmaceutically acceptable derivatives such as prodrugs, and salts and esters of the inventive compound.

The composition may be in any suitable form such as solid, semisolid, or liquid form. See Pharmaceutical Dosage Forms and Drug Delivery Systems, 5^{th} edition, Lippicott Williams & Wilkins (1991) which is incorporated herein by reference. In general, the pharmaceutical preparation will contain one or more of the compounds of the invention as an active ingredient in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used.

In one embodiment, the compositions containing an inventive compound are Cremophor®-free. Cremophor® (BASF Aktiengesellschaft) is a polyethoxylated castor oil which is typically used as a surfactant in formulating low soluble drugs. However, because Cremophor® can case allergic reactions in a subject, compositions that minimize or eliminate Cremophor® are preferred. Formulations of epothilone A or B that eliminate Cremophor® are described for example by PCT Publication WO 99/39694 which is incorporated herein by reference and may be adapted for use with the inventive compounds.

Where applicable, an inventive compound may be formulated as microcapsules and nanoparticles. General protocols are described for example, by Microcapsules and Nanoparticles in Medicine and Pharmacy by Max Donbrow, ed., CRC Press (1992) and by U.S. Pat. Nos. 5,510,118; 5,534,270; and 5,662,883 which are all incorporated herein by reference. By increasing the ratio of surface area to volume, these formulations allow for the oral delivery of compounds that would not otherwise be amenable to oral delivery.

An inventive compound may also be formulated using other methods that have been previously used for low solubility drugs. For example, the compounds may form emulsions with vitamin E or a PEGylated derivative thereof as described by PCT Publications WO 98/30205 and WO 00/71163 which are incorporated herein by reference. Typically, the inventive compound is dissolved in an aqueous solution containing ethanol (preferably less than 1% w/v). Vitamin E or a PEGylated-vitamin E is added. The ethanol is then removed to form a pre-emulsion that can be formulated for intravenous or oral routes of administration. Another strategy involves encapsulating the inventive compounds in liposomes. Methods for forming liposomes as drug delivery vehicles are well known in the art. Suitable protocols include those described by U.S. Pat. Nos. 5,683,715; 5,415,869, and 5,424,073 which are incorporated herein by reference, relating to another relatively low solubility cancer drug taxol and by PCT Publication WO 01/10412, which is incorporated herein by reference, relating to epothilone B. Of the various lipids that may be used, particularly preferred lipids for making epothilone-encapsulated liposomes include phosphatidylcholine and polyethyleneglycol-derivitized distearyl phosphatidylethanolamine.

Yet another method involves formulating an inventive compound using polymers such as polymers such as biopolymers or biocompatible (synthetic or naturally occurring) polymers. Biocompatible polymers can be categorized as biodegradable and non-biodegradable. Biodegradable polymers degrade in vivo as a function of chemical composition, method of manufacture, and implant structure. Illustrative examples of synthetic polymers include polyanhydrides, polyhydroxyacids such as polylactic acid, polyglycolic acids and copolymers thereof, polyesters polyamides polyorthoesters and some polyphosphazenes. Illustrative examples of naturally occurring polymers include proteins and polysaccharides such as collagen, hyaluronic acid, albumin, and gelatin.

Another method involves conjugating a compound of the present invention to a polymer that enhances aqueous solubility. Examples of suitable polymers include polyethylene glycol, poly-(d-glutamic acid), poly-(l-glutamic acid), poly-(l-glutamic acid), poly-(d-aspartic acid), poly-(l-aspartic acid), poly-(l-aspartic acid) and copolymers thereof. Polyglutamic acids having molecular weights between about 5,000 to about 100,000 are preferred, with molecular weights between about 20,000 and 80,000 being more preferred and with molecular weights between about 30,000 and 60,000 being most preferred. The polymer is conjugated via an ester linkage to one or more hydroxyls of an inventive epothilone using a protocol as essentially described by U.S. Pat. No. 5,977,163 which is incorporated herein by reference. Preferred conjugation sites include the hydroxyl off carbon-21 in the case of 21-hydroxy-epothilones. Other conjugation sites include, for example, the hydroxyl off carbon 3 and the hydroxyl off carbon 7.

In another method, an inventive compound is conjugated to a monoclonal antibody. This strategy allows the targeting of the inventive compound to specific targets. General protocols for the design and use of conjugated antibodies are described in Monoclonal Antibody-Based Therapy of Cancer by Michael L. Grossbard, ed. (1998), which is incorporated herein by reference.

The amount of active ingredient that may be combined with the carrier materials to produce a single dosage form will vary depending upon the subject treated and the particular mode of administration. For example, a formulation for intravenous use comprises an amount of the inventive compound ranging from about 1 mg/mL to about 25 mg/mL, preferably from about 5 mg/mL to 15 mg/mL, and more preferably about 10 mg/mL. Intravenous formulations are typically diluted between about 2 fold and about 30 fold with normal saline or 5% dextrose solution prior to use.

Methods of Treating Cancer

In one aspect of the present invention, the inventive compounds are used to treat cancer. In one embodiment, the compounds of the present invention are used to treat cancers of the head and neck which include tumors of the head, neck, nasal cavity, paranasal sinuses, nasopharynx, oral cavity, oropharynx, larynx, hypopharynx, salivary glands, and paragangliomas. In another embodiment, the compounds of the present invention are used to treat cancers of the liver and biliary tree, particularly hepatocellular carcinoma. In another embodiment, the compounds of the present invention are used to treat intestinal cancers, particularly colorectal cancer. In another embodiment, the compounds of the present invention are used to treat ovarian cancer. In another embodiment, the compounds of the present invention are used to treat small cell and non-small cell lung cancer. In another embodiment, the compounds of the present invention are used to treat breast cancer. In another embodiment, the compounds of the present invention are used to treat sarcomas which includes fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma. In another embodiment, the compounds of the present invention are used to treat neoplasms of the central nervous systems, particularly brain cancer. In another embodiment, the compounds of the present invention are used to treat lymphomas which include Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma.

The method comprises administering a therapeutically effective amount of an inventive compound to a subject suffering from cancer. The method may be repeated as necessary either to mitigate (i.e. prevent further growth) or to eliminate the cancer. Clinically, practice of the method will result in a reduction in the size or number of the cancerous growth and/or a reduction in associated symptoms (where applicable). Pathologically, practice of the method will produce at least one of the following: inhibition of cancer cell proliferation, reduction in the size of the cancer or tumor, prevention of further metastasis, and inhibition of tumor angiogenesis.

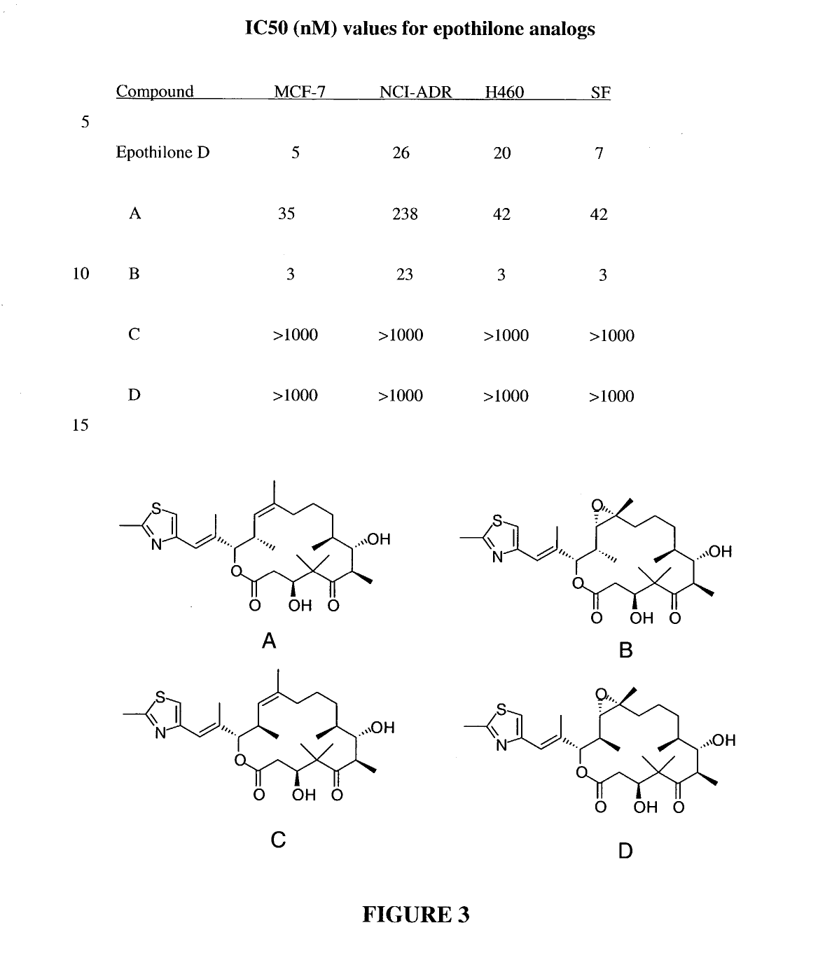

Cytotoxicity measurements in cell culture (FIG. 3) indicate that (14S)-14-methylepothilone D, the compound of formula (I) wherein R¹ is Me; R² is Me; R³ is Me; R⁴ is H; W is O; X is S; and Y is a bond, and (14R)-14-methylepothilone B, the compound of formula (I) wherein R¹ is Me; R² is Me; R³ is Me; R⁴ is H; W is O; X is S; and Y is O, are cytotoxic against MCF-7, NCI-ADR, H460, and SF cancer cell lines. Both (14R)-14-methylepothilone D and (14S)-14-methylepothilone B, the corresponding diastereomers wherein R³ is H and R⁴ is Me, appear to be essentially inactive in this assay.

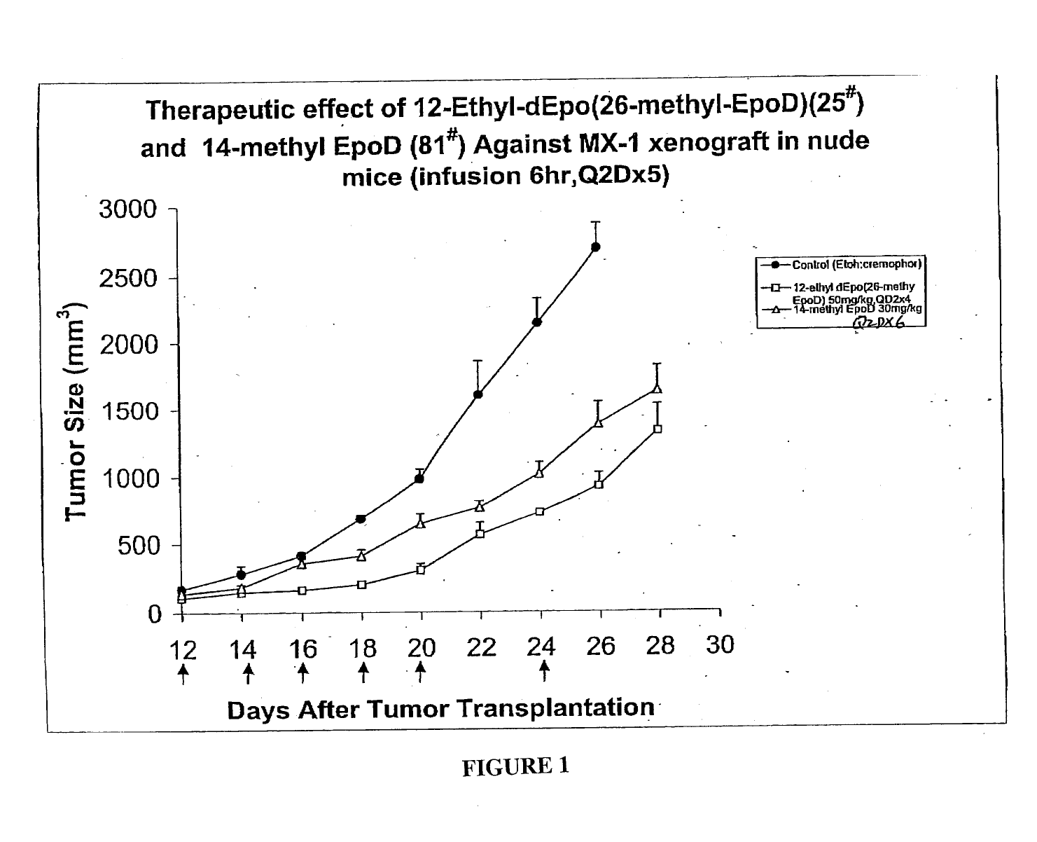

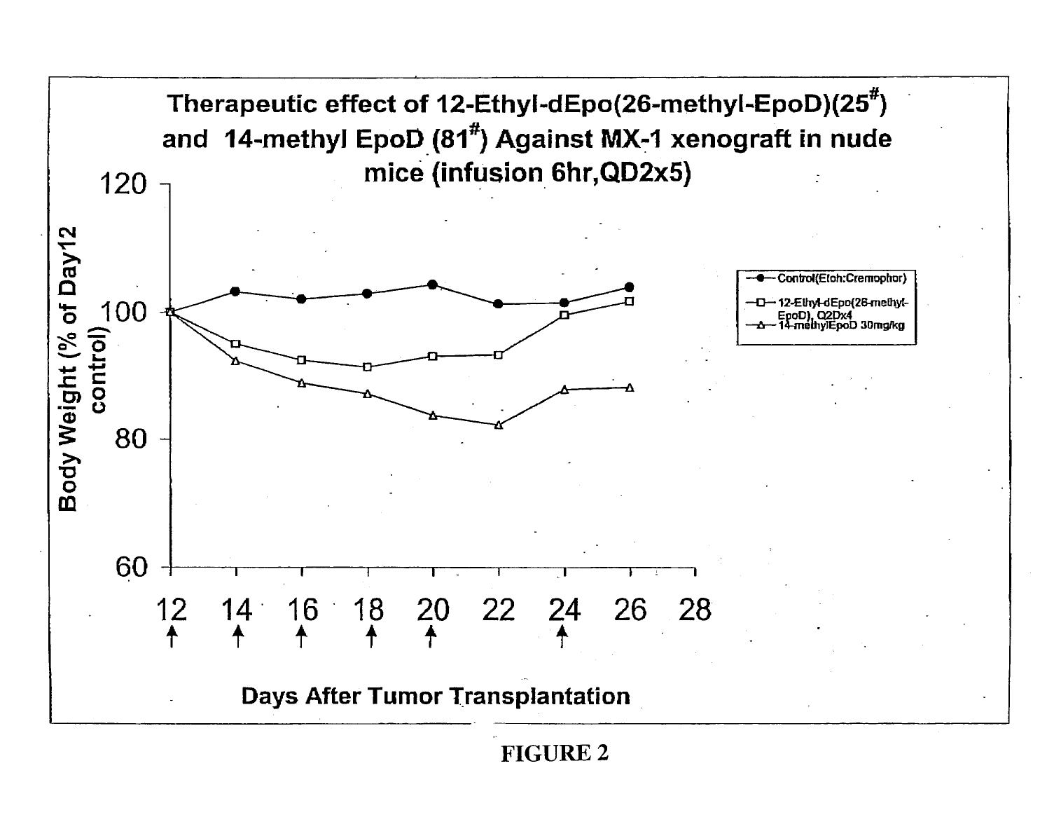

As shown in FIG. 1, treatment of nude mice having the MX-1 tumor xenograft with an infusion of (14S)-14-methylepothilone D twice daily for five days results in a decrease in the rate of tumor growth compared with the control (pharmaceutical carrier only) as measured by the tumor size. As shown in FIG. 2, this treatment results in a slight decrease in body weight during the course of treatment.

The compounds and compositions of the present invention can be used in combination therapies. In other words, the inventive compounds and compositions can be administered concurrently with, prior to, or subsequent to one or more other desired therapeutic or medical procedures. The particular combination of therapies and procedures in the combination regimen will take into account compatibility of the therapies and/or procedures and the desired therapeutic effect to be achieved.

In one embodiment, the compounds and compositions of the present invention are used in combination with another anti-cancer agent or procedure. Illustrative examples of other anti-cancer agents include but are not limited to: (i) alkylating drugs such as mechlorethamine, chlorambucil, cyclophosphamide, melphalan, ifosfamide; (ii) antimetabolites such as methotrexate; (iii) microtubule stabilizing agents such as vinblastin, paclitaxel, docetaxel, and discodermolide; (iv) angiogenesis inhibitors; (v) and cytotoxic antibiotics such as doxorubicin (adriamycin), bleomycin, and mitomycin. Illustrative examples of other anti-cancer procedures include: (i) surgery; (ii) radiotherapy; and (iii) photodynamic therapy.

In another embodiment, the compounds and compositions of the present invention are used in combination with an agent or procedure to mitigate potential side effects from the inventive compound or composition such as diarrhea, nausea and vomiting. Diarrhea may be treated with antidiarrheal agents such as opioids (e.g. codeine, diphenoxylate, difenoxin, and loeramide), bismuth subsalicylate, and octreotide. Nausea and vomiting may be treated with antiemetic agents such as dexamethasone, metoclopramide, diphenyhydramine, lorazepam, ondansetron, prochlorperazine, thiethylperazine, and dronabinol. For those compositions that includes polyethoxylated castor oil such as Cremophor®, pretreatment with corticosteroids such as dexamethasone and methylprednisolone and/or H₁ antagonists such as diphenylhydramine HCl and/or H₂ antagonists may be used to mitigate anaphylaxis.

Methods of Treating of Non-cancer, Cellular Hyperproliferative Disorders

In another aspect of the present invention, the inventive compounds are used to treat non-cancer disorders that are characterized by cellular hyperproliferation (e.g., an abnormally increased rate or amount of cellular proliferation). In one embodiment, the compounds of the present invention are used to treat psoriasis, a condition characterized by the cellular hyperproliferation of keratinocytes which builds up on the skin to form elevated, scaly lesions. The method comprises administering a therapeutically effective amount of an inventive compound to a subject suffering from psoriasis. The method may be repeated as necessary either to decrease the number or severity of lesions or to eliminate the lesions. Clinically, practice of the method will result in a reduction in the size or number of skin lesions, diminution of cutaneous symptoms (pain, burning and bleeding of the affected skin) and/or a reduction in associated symptoms (e.g., joint redness, heat, swelling, diarrhea, abdominal pain). Pathologically, practice of the method will result in at least one of the following: inhibition of keratinocyte proliferation, reduction of skin inflammation (for example, by impacting on: attraction and growth factors, antigen presentation, production of reactive oxygen species and matrix metalloproteinases), and inhibition of dermal angiogenesis.

In another embodiment, the compounds of the present invention are used to treat multiple sclerosis, a condition characterized by progressive demyelination in the brain. Although the exact mechanisms involved in the loss of myelin are not understood, there is an increase in astrocyte proliferation and accumulation in the areas of myelin destruction. At these sites, there is macrophage-like activity and increased protease activity which is at least partially responsible for degradation of the myelin sheath. The method comprises administering a therapeutically effective amount of an inventive compound to a subject suffering from multiple sclerosis. The method may be repeated as necessary to inhibit astrocyte proliferation and/or lessen the severity of the loss of motor function and/or prevent or attenuate chronic progression of the disease. Clinically, practice of the method will result in improvement in visual symptoms (visual loss, diplopia), gait disorders (weakness, axial instability, sensory loss, spasticity, hyperreflexia, loss of dexterity), upper extremity dysfunction (weakness, spasticity, sensory loss), bladder dysfunction (urgency, incontinence, hesitancy, incomplete emptying), depression, emotional lability, and cognitive impairment. Pathologically, practice of the method will result in the reduction of one or more of the following, such as myelin loss, breakdown of the blood-brain barrier, perivascular infiltration of mononuclear cells, immunologic abnormalities, gliotic scar formation and astrocyte proliferation, metalloproteinase production, and impaired conduction velocity.

In another embodiment, the compounds of the present invention are used to treat rheumatoid arthritis, a multisystem chronic, relapsing, inflammatory disease that sometimes leads to destruction and ankyiosis of affected joints. Rheumatoid arthritis is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and, eventually the pannus grows to fill the joint space. The pannus develops an extensive network of new blood vessels through the process of angiogenesis that is essential to the evolution of the synovitis. Release of digestive enzymes (matrix metalloproteinases (e.g., collagenase, stromelysin)) and other mediators of the inflammatory process (e.g., hydrogen peroxide, superoxides, lysosomal enzymes, and products of arachadonic acid metabolism) from the cells of the pannus tissue leads to the progressive destruction of the cartilage tissue. The pannus invades the articular cartilage leading to erosions and fragmentation of the cartilage tissue. Eventually there is erosion of the subchondral bone with fibrous ankylosis and ultimately bony ankylosis, of the involved joint.

The method comprises administering a therapeutically effective amount of an inventive compound to a subject suffering from rheumatoid arthritis. The method may be repeated as necessary to accomplish to inhibit synoviocyte proliferation and/or lessen the severity of the loss of movement of the affected joints and/or prevent or attenuate chronic progression of the disease. Clinically, practice of the present invention will result in one or more of the following: (i) decrease in the severity of symptoms (pain, swelling and tenderness of affected joints; morning stiffness. weakness, fatigue. anorexia, weight loss); (ii) decrease in the severity of clinical signs of the disease (thickening of the joint capsule. synovial hypertrophy, joint effusion, soft tissue contractures, decreased range of motion, ankylosis and fixed joint deformity); (iii) decrease in the extra-articular manifestations of the disease (rheumatic nodules, vasculitis, pulmonary nodules, interstitial fibrosis, pericarditis, episcleritis, iritis, Felty's syndrome, osteoporosis); (iv) increase in the frequency and duration of disease remission/symptom-free periods; (v) prevention of fixed impairment and disability; and/or (vi) prevention/attenuation of chronic progression of the disease. Pathologically, practice of the present invention will produce at least one of the following: (i) decrease in the inflammatory response; (ii) disruption of the activity of inflammatory cytokines (such as IL-I, TNFa, FGF, VEGF); (iii) inhibition of synoviocyte proliferation; (iv) inhibition of matrix metalloproteinase activity, and/or (v) inhibition of angiogenesis.

In another embodiment, the compounds of the present invention are used to prevent cellular proliferation on a prosthesis implanted in a subject by coating the prosthesis with a composition containing a compound of the present invention. In another embodiment, compounds of the present invention are used to treat atherosclerosis and/or restenosis, particularly in patients whose blockages may be treated with an endovascular stent. Atherosclerosis is a chronic vascular injury in which some of the normal vascular smooth muscle cells (“VSMC”) in the artery wall, which ordinarily control vascular tone regulating blood flow, change their nature and develop “cancer-like” behavior. These VSMC become abnormally proliferative, secreting substances (growth factors, tissue-degradation enzymes and other proteins) which enable them to invade and spread into the inner vessel lining, blocking blood flow and making that vessel abnormally susceptible to being completely blocked by local blood clotting. Restenosis, the recurrence of stenosis or artery stricture after corrective procedures, is an accelerated form of atherosclerosis.

The method comprises coating a therapeutically effective amount of an inventive compound on a stent and delivering the stent to the diseased artery in a subject suffering from atherosclerosis. Methods for coating a stent with a compound are described for example by U.S. Pat. Nos. 6,156,373 and 6,120,847. Clinically, practice of the present invention will result in one or more of the following: (i) increased arterial blood flow; (ii) decrease in the severity of clinical signs of the disease; (iii) decrease in the rate of restenosis; or (iv) prevention/attenuation of the chronic progression of atherosclerosis. Pathologically, practice of the present invention will produce at least one of the following at the site of stent implanataion: (i) decrease in the inflammatory response, (ii) inhibition of VSMC secretion of matrix metalloproteinases; (iii) inhibition of smooth muscle cell accumulation; and (iv) inhibition of VSMC phenotypic dedifferentiation.

Dosage Levels

In one embodiment, dosage levels that are administered to a subject suffering from cancer or a non-cancer disorder characterized by cellular proliferation are of the order from about 1 mg/m² to about 200 mg/m² which may be administered as a bolus (in any suitable route of administration) or a continuous infusion (e.g. 1 hour, 3 hours, 6 hours, 24 hours, 48 hours or 72 hours) every week, every two weeks, or every three weeks as needed. It will be understood, however, that the specific dose level for any particular patient depends on a variety of factors. These factors include the activity of the specific compound employed; the age, body weight, general health, sex, and diet of the subject; the time and route of administration and the rate of excretion of the drug; whether a drug combination is employed in the treatment; and the severity of the condition being treated.

In another embodiment, the dosage levels are from about 10 mg/m² to about 150 mg/m², preferably from about 10 to about 75 mg/m² and more preferably from about 15 mg/m² to about 50 mg/m² once every three weeks as needed and as tolerated. In another embodiment, the dosage level is about 13 mg/m² once every three weeks as needed and as tolerated. In another embodiment, the dosage levels are from about 1 mg to about 150 mg/m², preferably from about 10 mg/m² to about 75 mg/m² and more preferably from about 25 mg/m² to about 50 mg/m² once every two weeks as needed and as tolerated. In another embodiment, the dosage levels are from about 1 mg/m² to about 100 mg/m², preferably from about 5 mg/m² to about 50 mg/m² and more preferably from about 10 mg/m² to about 25 mg/m² once every week as needed and as tolerated. In another embodiment, the dosage levels are from about 0.1 to about 25 mg/m², preferably from about 0.5 to about 15 mg/m² and more preferably from about 1 mg/m² to about 10 mg/m² once daily as needed and tolerated.

A detailed description of the invention having been provided above, the following examples are given for the purpose of illustrating the present invention and shall not be construed as being a limitation on the scope of the invention or claims.

### EXAMPLE 1

### (3S,4S)-2-ethoxy-3-hydroxy-4-methyl-5-(4-methoxybenzyloxy)-1-pentene

A 1.7 M solution of tert-butyllithium in pentane (100 mL) is added to a solution of ethyl vinyl ether (20 g) in 200 mL of THF under inert atmosphere at −78° C. After stirring for 1 hour, a solution of magnesium bromide diethyl etherate (51.6 g) in 500 mL of THF is added over 30 minutes, and the solution is stirred an additional 1 hour. A solution of (2S)-2-methyl-3-(4-methoxybenzyloxy)propanal (Smith et al., J. Am. Chem. Soc. (2000) 122: 8654-8664) (35 g) in 100 mL of THF is added over 1 hour. After an additional 1 hour, the mixture is warmed to ambient temperature, poured into sat. NH₄Cl and extracted with ether. The extract is washed with water and brine, then dried over MgSO₄, filtered, and evaporated. The product is purified by silica gel chromatography.

### EXAMPLE 2

### (3S,4S)-2-ethoxy-3-(tert-butyldimethylsilyloxy)-4-methyl-5-(4-methoxybenzyloxy)-1-pentene

A mixture of (3S,4S)-2-ethoxy-3-hydroxy-4-methyl-5-(4-methoxybenzyloxy)-1-pentene (28 g) and imidazole (7.0 g) in 200 mL of dimethylformamide is treated with tert-butyldimethylsilyl chloride (16.0 g) for 12 hours at ambient temperature. The mixture is poured into water and extracted with 1:1 ether/hexane. The extract is washed successively with water and brine, then dried over MgSO₄, filtered, and evaporated. The product is purified by silica gel chromatography.

### EXAMPLE 3

### (3S,4S)-2-ethoxy-3-(tert-butyldimethylsilyloxy)-4-methyl-5-hydroxy-1-pentene

A solution of (3S,4S)-2-ethoxy-3-(tert-butyldimethylsilyloxy)-4-methyl-5-(4-methoxybenzyloxy)-1-pentene (5.1 g) in 125 mL of CH₂Cl₂ is treated with water (6 mL) and 2,3-dichloro-5,6-dicyano-1,4-benzoquinone (3.2 g) for 3 hours. Saturated NaHCO₃ is added (20 mL), and the phases are separated. The organic phase is dried over MgSO₄, filtered, and concentrated. The product is purified by chromatography on silica gel.

### EXAMPLE 4

### (3S,4S)-2-ethoxy-3-(tert-butyldimethylsilyloxy)-4-methyl-5-oxo-1-pentene

A solution of DMSO (7.2 mL) in 150 mL of CH₂Cl₂ is cooled to −78° C. and treated with oxalyl chloride (4.4 mL) over 30 minutes. After an additional 30 minutes, a solution of (3S,4S)-2-ethoxy-3-(tert-butyldimethylsilyloxy)-4-methyl-5-hydroxy-1-pentene (8.6 g) in 10 mL of CH₂Cl₂ is added dropwise over 30 minutes. After an additional 45 minutes, diisopropylethylamine (34.5 mL) is added over 45 minutes. The mix is stirred for 30 minutes, then is allowed to warm to ambient temperature and poured into 200 mL of vigorously stirred 1.0 M NaHSO₄. The phases are separated, and the aqueous phase is extracted with ether. The extract is combined with the organic phase and concentrated. The residue is dissolved in ether and washed sequentially with aq. NaHSO₄, water, sat. NaHCO₃, and brine, then dried over MgSO₄, filtered, and evaporated to provide the product aldehyde.

### EXAMPLE 5

### (3S,4S,5Z)-2-ethoxy-3-(tert-butyldimethylsilyloxy)-4-methyl-6-iodo-1,5-heptadiene

A suspension of dry ethyl triphenylphosphonium iodide (68.7 g) in 600 mL of THF is treated with 2.5 M n-butyllithium in hexane (64 mL) over 30 minites. After an additional 10 minutes, the red solution is added via cannula to a −78° C. solution of iodine (41.7 g) in 1400 mL of THF at such a rate that the internal temperature remains below −70° C. The yellow slurry is warmed to −20° C., and 1.0 M sodium hexamethyldisilazide in THF (147 mL) is added over 30 minutes. After an additional 15 minutes, the orange solution is cooled to −33° C., and a solution of (3S,4S)-2-ethoxy-3-(tert-butyldimethylsilyloxy)-4-methyl-5-oxo-1-pentene (22.5 g) in 200 mL of THF is added over 15 minutes. The mix is stirred for 45 minutes, then warmed to ambient temperature, quenched by addition of 20 mL of methanol, and concentrated. The residue is filtered through silica gel using ether, and the eluate is washed successively with sat. Na₂S₂O₃ and brine, dried over MgSO₄, filtered, and concentrated. The product is purified by chromatography on silica gel.

### EXAMPLE 6

### (3S,4S,5Z)-3-(tert-butyldimethylsilyloxy)-4-methyl-6-iodo-hept-5-en-2-one

A solution of (3S,4S,5Z)-2-ethoxy-3-(tert-butyldimethylsilyloxy)-4-methyl-6-iodo-1,5-heptadiene (41.0 g) in 400 mL of acetone and 100 mL of 0.1 N HCl is stirred at ambient temperature until consumption of starting material as determined by thin layer chromatographic analysis. The mixture is neutralized by addition of sat. NaHCO₃ and concerntrated to an aqueous slurry, which is extracted with ether. The extract is washed with brine, dried over MgSO₄, filtered, and concentrated. The product is purified by chromatography on silica gel.

### EXAMPLE 7

### (4S,5S,2Z,6E)-5-(tert-butyldimethylsilyloxy)-4,6-dimethyl-2-iodo-7-(2-methythiazol-4-yl)-hepta-2,6-diene

A 1.0 M solution of sodium hexamethyldisilazide in tetrahydrofuran (18 mL) is added dropwise to a −78° C. solution of (2-methythiazol-4-yl)methyl diphenylphosphine oxide (6.90 g) and (3S,4S,5Z)-3-(tert-butyldimethylsilyloxy)-4-methyl-5-iodo-hept-5-en-2-one (5.65 g) in 15 mL of THF. The mix is allowed to warm to ambient temperature, stirred for 10 hours, then is poured into sat. NH₄Cl and extracted with ether. The extract is washed sequentially with sat. NaHCO₃ and brine. The solution is dried over MgSO₄, filtered, and evaporated. The product is purified by silica gel chromatography.

### EXAMPLE 8

### (3Z,5S,6S,7E)-6-(tert-butyldimethylsilyloxy)-8-(2-methylthiazol-4-yl)-3,5,7-trimethyl-octa-1,3,7-triene

A mixture of tetrakis(triphenylphosphine)palladium (1.18 g) and lithium chloride (12.9 g) in 500 mL of THF is stirred under argon for 15 minutes, then a solution of (4S,5S,2Z,6E)-5-(tert-butyldimethylsilyloxy)-4,6-dimethyl-2-iodo-7-(2-methythiazol-4-yl)-hepta-2,6-diene (46.7 g) and vinyltributylstannane (31.6 g) in 250 mL of THF is added followed by an additional 25 mL of THF. The resulting solution is heated at reflux for 48 hours, then cooled and partitioned between 500 mL water and 250 mL of pentane. The aqueous phase is extracted with pentane, and the the extract is combined with the original organic phase and washed sequentially with sat. NaHCO₃ and brine. The solution is dried over MgSO₄, filtered, and evaporated. The product is purified by silica gel chromatography.

### EXAMPLE 9

### (3Z,5S,6S,7E)-6-hydroxy-8-(2-methylthiazol-4-yl)-3,5,7-trimethyl-octa-1,3,7-triene

A solution of (3Z,5S,6S,7E)-6-(tert-butyldimethylsilyloxy)-8-(2-methylthiazol-4-yl)-3,5,7-trimethyl-octa-1,3,7-triene (3.8 g) in 100 mL of acetonitrile is cooled on ice and treated dropwise with 5 mL of 48% hydrofluoric acid. After stirring for 1 hour, the mixture is quenched by careful addition of sat. NaHCO₃ and extracted with ethyl acetate. The extract is washed with brine, dried over MgSO₄, filtered, and evaporated. The product is purified by silica gel chromatography.

### EXAMPLE 10

### tert-butyl (3S,6R,7S,8S)-5-oxo-3-(triethylsilyloxy)-4,4,6,8-tetramethyl-7-(2,2,2-trichloroethoxycarbonyloxy)-10-undecenoate

(a) (4R,5S,6S)-1,1-diisopropoxy-5-hydroxy-2,2,4,6-tetramethyl-8-nonen-3-one. A solution of 1,1-diisopropoxy-2,2,-dimethyl-3-pentanone (3.29 g) in 15 mL of THF is added slowly to a solution of lithium diisopropylamide (15.7 mmol) in 20 mL of THF cooled to −78° C., the mixture is stirred for 30 minutes, warmed to −40° C. and stirred for 30 minutes, then recooled to −78° C. A solution of (2S)-2-methyl-4-pentenal (16.36 mmol) in 2 mL of CH₂Cl₂ is added and the mixture is stirred for 1 hour at −78° C. Saturated aq. NH₄Cl is added and the mixture is warmed to ambient temperature and extracted with ethyl acetate. The extract is dried over Na₂SO₄, filtered, and evaporated. The residue is purified by silica gel chromatography (2% ethyl acetate/hexanes) to separate the two diastereomeric products.

(4R,5S,6S)-1,1-diisopropoxy-5-(2,2,2-trichloroethoxycarbonyloxy)-2,2,4,6-tetramethyl-8-nonen-3-one. Trichloroethyl chloroformate (2.5 mL) and pyridine (2.95 mL) are added to a solution of (4R,5S,6S)-1,1-diisopropoxy-5-hydroxy-2,2,4,6-tetramethyl-8-nonen-3-one (3.0 g) in 40 mL of CH₂Cl₂ at 0° C., and the mixture is stirred for 5 hours before pouring into sat. aq. NaCl and extracting with CH₂Cl₂. The extract is dried over Na₂SO₄, filtered, and evaporated. The product is purified by chromatography on SiO₂ (2% ethyl acetate/hexanes).

(4R,5S,6S)-3-oxo-5-(2,2,2-trichloroethoxycarbonyloxy)-2,2,4,6-tetramethyl-8-nonenal. A mixture of (4R,5S,6S)-1,1-diisopropoxy-5-(2,2,2-trichloroethoxy-carbonyloxy)-2,2,4,6-tetramethyl-8-nonen-3-one (4.58 g) and p-toluenesulfonic acid monohydrate (0.45 g) in 100 mL of 3:1 THF/water is heated at reflux for 7 hours. The mixture is cooled and poured into sat. aq. NaHCO₃, then extracted with ethyl acetate. The extract is dried over Na₂SO₄, filtered, and evaporated. The product is purified by chromatography on SiO₂ (3% ethyl acetate/hexanes).

tert-butyl (3S,6R,7S,8S)-5-oxo-3-hydroxy-4,4,6,8-tetramethyl-7-(2,2,2-trichloroethoxycarbonyloxy)-10-undecenoate. Tert-butyl acetate (0.865 mL) is added to a solution of lithium diisopropylamide (7.52 mmol) in 30 mL of ether at −78° C., and the mixture is stirred for 1 hour. A solution of bis(1,2:5,6-di-O-isopropylidene-_-L-glucofuranos-3-O-yl)cyclopentadienyltitanium chloride (8.34 mmol) in 90 mL of ether is added dropwise over 40 minutes, and the reaction is stirred for an additional 30 minutes at −78° C., warmed to −30° C. and kept for 45 minutes, then recooled to −78° C. A solution of (4R,5S,6S)-3-oxo-5-(2,2,2-trichloroethoxycarbonyloxy)-2,2,4,6-tetramethyl-8-nonenal (2.57 g) in 15 mL of ether is added over 10 minutes and the reaction is continued for 2 hours before addition of 14 mL of 5 M water in THF. The mix is stirred for 1 hour, then filtered through Celite. The filtrate is washed with sat. aq. NaCl, and the brine layer is back extracted with ether. The organic phases are combined, dried with Na₂SO₄, filtered, and evaporated. The product is purified by chromatography on SiO₂ (7% ethyl acetate/hexanes).

Tert-butyl (3S,6R,7S,8S)-5-oxo-3-(triethylsilyloxy)-4,4,6,8-tetramethyl-7-(2,2,2-trichloroethoxycarbonyloxy)-10-undecenoate. A solution of tert-butyl (3S,6R,7S,8S)-5-oxo-3-hydroxy-4,4,6,8-tetramethyl-7-(2,2,2-trichloroethoxycarbonyloxy)-10-undecenoate (1.8 g), imidazole (0.48 g), and triethylsilyl chloride (0.68 g) in 5 mL of dimethylformamide is stirred for 2 hours at ambient temperature, then poured into water and extracted with ether. The extract is washed with sat. aq. NaCl, dried over MgSO₄, filtered, and evaporated. The product is purified by chromatography on SiO₂ (20:1 toluene/ethyl acetate).

### EXAMPLE 11

### (3S,6R,7S,8S)-5-oxo-3-(triethylsilyloxy)-4,4,6,8-tetramethyl-7-(2,2,2-trichloroethoxycarbonyloxy)-10-undecenoic acid

A solution of tert-butyl (3S,6R,7S,8S)-5-oxo-3-(triethylsilyloxy)-4,4,6,8-tetramethyl-7-(2,2,2-trichloroethoxycarbonyloxy)-10-undecenoate (6.3 g) and 2,6-lutidine (14 mL) in 200 mL of CH₂Cl₂ is cooled to −78° C. and treated with triethylsilyl triflate (13.7 mL). The mixture is stirred 12 hours, then warmed to ambient temperature and quenched by addition of 400 mL of sat. NH₄Cl and poured into 500 mL of CH₂Cl₂. The phases are separated, and the organic phase is washed with pH 7 phosphate buffer and concentrated. The residue is dissolved in 100 mL of THF, cooled on ice, and treated with 0.12 M HCL in methanol (100 mL). After 20 minutes, the reaction is quenched with sat. NaHCO₃ and extracted with ethyl acetate. The extract is dried over MgSO₄, filtered, and evaporated. The product is purified by silica gel chromatography.

### EXAMPLE 12

### (3Z,5S,6S,7E)-8-(2-methylthiazol-4-yl)-3,5,7-trimethyl-octa-1,3,7-trien-6-yl (3S,6R,7S,8S)-5-oxo-3-(triethylsilyloxy)-4,4,6,8-tetramethyl-7-(2,2,2-trichloroethoxycarbonyloxy)-10-undecenoate

A solution of (3S,6R,7S,8S)-5-oxo-3-(triethylsilyloxy)-4,4,6,8-tetramethyl-7-(2,2,2-trichloroethoxycarbonyloxy)-10-undecenoic acid (5.76 g), (3Z,5S,6S,7E)-6-hydroxy-8-(2-methylthiazol-4-yl)-3,5,7-trimethyl-octa-1,3,7-triene (2.63 g), 4-(dimethylamino)pyridine (1.2 g), and 1-[3-(dimethylamino)propyl]-3-ethylcarbodiimide hydrochloride (2.5 g) in 100 mL of CH₂Cl₂ is stirred at ambient temperature for 12 hours, then washed sequentially with water, sat. NaHCO₃, and brine. The solution is dried over MgSO₄, filtered, and evaporated. The product is purified by silica gel chromatography.

### EXAMPLE 13

### (14S)-10,11-dehydro-14-methyl-7-O-(2,2,2-trichloroethoxycarbonyl)-3-O-triethylsilyl-epothilone D

A solution of (3Z,5S,6S,7E)-8-(2-methylthiazol-4-yl)-3,5,7-trimethyl-octa-1,3,7-trien-6-yl (3S,6R,7S,8S)-5-oxo-3-(triethylsilyloxy)-4,4,6,8-tetramethyl-7-(2,2,2-trichloroethoxycarbonyloxy)-10-undecenoate (6.0 g) and tricyclohexylphosphine[1,3-bis(2,4,6-trimethylphenyl)-4,5-dihydroimidazol-2-ylidene][benzylidene]-ruthenium(IV)dichloride (2 g) in 100 mL of benzene is stirred for 24 hours, then concentrated. The product is purified by chromatography on silica gel.

### EXAMPLE 14

### (14S)-10,11-dehydro-14-methyl-epothilone D

(a) A solution of (14S)-14-methyl-7-O-(2,2,2-trichloroethoxycarbonyl)-3-O-(triethylsilyl)-10,11-dehydroepothilone D (0.2 g) in 1 mL of THF is added to a stirred suspension of activated zinc dust (0.261 g) in 2 mL of acetic acid. After stirring for 1.5 hours, the mixture is diluted with ethyl acetate and filtered. The filtrate is washed sequentially with 10% NaHCO₃ and brine, dried over MgSO₄, filtered, and evaporated. The product is purified by flash chromatography on SiO₂ (2:1 hexanes/ethyl acetate).

(14S)-14-methyl-10,11-dehydroepothilone D. A solution of (14S)-14-methyl-3-O-(triethylsilyl)-10,11-dehydroepothilone D (80 mg) in 2 mL of THF in a polyethylene vessel and treated with 1.5 mL of HF.pyridine for 1 hour at 0° C. and 30 minutes at ambient temperature, then diluted with 30 mL of ethyl acetate and poured into 20 mL of sat. aq. NaHCO₃. The organic phase is separated and washed sequentially with 1 N HCl, 10% NaHCO₃, and brine, then dried over MgSO₄, filtered, and evaporated. The product is purified by flash chromatography on SiO₂ (1:2 hexanes/ethyl acetate).

### EXAMPLE 15

### (14S)-14-methyl-epothilone D

A mixture of (14S)-10,11-dehydro-14-methyl-epothilone D (500 mg), dipotassium azodicarboxylate (500 mg), and acetic acid (0.5 mL) in 10 mL of anhydrous dioxane is stirred on ice for 1 hour, then is poured into sat. NaHCO₃ and extracted with ethyl acetate. The extract is washed with brine, then dried over MgSO₄, filtered, and evaporated. The product is purified by flash chromatography on SiO₂ (1:2 hexanes/ethyl acetate).

### EXAMPLE 16

### 2-methyl-4-(chloromethyl)thiazole

A mixture of thioacetamide (6.9 g) and 1,3-dichloroacetone (13.4 g) in 100 mL of toluene is heated under reflux for 2 hours, then cooled to ambient temperature and washed sequentially with sat. NaHCO₃ and brine. The solution is dried over MgSO₄, filtered, and evaporated. The product is purified by silica gel chromatography.

### EXAMPLE 17

### (2-methythiazol-4-yl)methyl diphenylphosphine oxide

A mixture of 2-methyl-4-(chloromethyl)thiazole (6.0 g), diphenylphosphine oxide (9.1 g), cesium carbonate (16.3 g), 4 Å molecular sieves (ca. 0.5 g), and tetrabutylammonium iodide (0.15 g) in 60 mL of CH₂Cl₂ is stirred for 2 days. The mix is poured into sat. aq. NaHSO₄ and extracted with ethyl acetate. The extract is washed sequentially with sat. NaHCO₃ and brine. The solution is dried over MgSO₄, filtered, and evaporated. The product is purified by silica gel chromatography.

### EXAMPLE 18

### Ethyl O-(4-methoxybenzyl)-(S)-lactate

p-Methoxybenzyl alcohol (200 g) is added to a suspension of NaH (5.82 g of a 60% dispersion in oil) in 450 mL of anhydrous ether over 1 hour at ambient temperature. After an additional 1 hour, the mix is cooled on ice and treated with trichloroacetonitrile (158 mL) over 80 minutes. After an additional 1.5 hour the solution is concentrated at low temperature. The residue is treated with a mix of pentane (1500 mL) and methanol (5.6 mL), stirred for 30 minutes, then filtered through a short plug of Celite and concentrated to give 4-methoxybenzyl trichloroacetimidate.

A mixture of ethyl (S)-lactate (128 g) and 4-methoxybenzyl trichloroacetimidate (371 g) in 1:2 CH₂Cl₂/cyclohexane (1500 mL) is cooled on ice and treated with pyridinium p-toluenesulfonate (13.7 g). After 3 hours, the mixture is warmed to ambient temperature and kept 40 hours, then concentrated. The residue is filtered through a plug of silica gel using 20% ethyl acetate in hexanes and concentrated to yield the product.

### EXAMPLE 19

### O-(4-methoxybenzyl)-(S)-lactaldehyde

A solution of ethyl O-(4-methoxybenzyl)-(S)-lactate (116 g) in 800 mL of anhydrous THF is cooled to 0° C. and added via cannula to a 0.67 M solution of LiAlH₄ in THF (800 mL) over 1 hour. The mix is allowed to warm to ambient temperature and is stirred an additional 1 hour, then is cooled on ice and treated dropwise with water (20 mL), 15% NaOH (20 mL), and water (60 mL). The mix is treated with MgSO₄ (10 g), filtered, and concentrated to yield the intermediate alcohol.

A solution of DMSO (72.1 mL) in 1500 mL of CH₂Cl₂ is cooled to −78° C. and treated with oxalyl chloride (44.3 mL) over 30 minutes. After an additional 30 minutes, a solution of the intermediate alcohol from above (71.2 g) in 100 mL of CH₂Cl₂ is added dropwise over 30 minutes. After an additional 45 minutes, diisopropylethylamine (345 mL) is added over 45 minutes. The mix is stirred for 30 minutes, then is allowed to warm to ambient temperature and poured into 2000 mL of vigorously stirred 1.0 M NaHSO₄. The phases are separated, and the aqueous phase is extracted with ether. The extract is combined with the organic phase and concentrated. The residue is dissolved in ether and washed sequentially with aq. NaHSO₄, water, sat. NaHCO₃, and brine, then dried over MgSO₄, filtered, and evaporated to provide the product aldehyde.

### EXAMPLE 20

### (4R)-3-[(2S,3S,4S)-3-hydroxy-4-(4-methoxybenzyloxy)-2-methylpentanoyl]-4-benzyl-2-oxazolidinone

A solution of (4R)-4-benzyl-3-propionyl-2-oxazolidinone (91 g) in 972 mL of CH₂Cl₂ is cooled to −20° C. and treated with 1.0 M di-n-butylboron triflate in CH₂Cl₂ (403 mL) over 30 minutes, followed by triethylamine (61.3 mL) over 20 minutes. The mixture is warmed to 0° C., kept for 10 minutes, then cooled to −78° C. A degassed solution of O-(4-methoxybenzyl)-(S)-lactaldehyde (70.5 g) in 200 mL of CH₂Cl₂ is added over 1 hour. After an additional 1 hour, the mixture is warmed to −10° C., kept for 1 hour, then quenched by addition of 220 mL of 0.5 M phosphate buffer, pH 7. A solution of 30% hydrogen peroxide (230 mL) and 470 mL of methanol is added at such a rate as to keep the internal temperature below −10° C. with vigorous stirring. The mix is warmed to ambient temperature and stirred for 10 hours, then concentrated to ca. 1000 mL. The residue is dissolved in 1500 mL of 10:1 ether/CH₂Cl₂ and the phases are separated. The aqueous phase is extracted with 10:1 ether/CH₂Cl₂ and the extract is combined with the organic phase. The combined extracts are washed sequentially with sat. NaHCO₃, water, and brine, then dried over MgSO₄, filtered, and evaporated. The product is purified by crystallization.

### EXAMPLE 21

### N-methoxy N-methyl (2S,3S,4S)-3-hydroxy-4-(4-methoxybenzyloxy)-2-methylpentanamide

A suspension of N,O-dimethylhydroxylamine hydrochloride (50.8 g) in 380 mL of THF is cooled on ice and treated cautiously with 2.0 M trimethylaluminum in hexane (256 mL) over 30 minutes. After stirring for 30 minutes on ice and 90 minutes at ambient temperature, the solution is cooled to −20° C. and a solution of (4R)-3-[(2S,3S,4S)-3-hydroxy-4-(4-methoxybenzyloxy)-2-methylpentanoyl]-4-benzyl2-oxazolidinone (74.4 g) in 380 mL of THF is added over 60 minutes via cannula. After 90 minutes, the solution is carefully poured into a mix of 1.0 N HCl (1000 mL) and CH₂Cl₂ (1000 mL) and stirred vigourously for 90 minutes. The phases are separated, the aqueous phase is extracted with CH₂Cl₂, and the extract is combined with the organic phase. The combined extracts are washed sequentially with water and brine, then dried over MgSO₄, filtered, and evaporated. The product is purified by crystallization.

### EXAMPLE 22

### N-methoxy N-methyl (2S,3S,4S)-3-(tert-butyldimethylsilyloxy)-4-(4-methoxybenzyloxy)-2-methylpentanamide

A solution of N-methoxy N-methyl (2S,3S,4S)-3-hydroxy-4-(4-methoxybenzyloxy)-2-methlylpentanamide (31.1 g) in 500 mL of CH₂Cl₂ at 0° C. is treated with 2,6-lutidine (20 g) and tert-butyldimethylsilyl triflate (28 g). The mix is kept for 12 hours at ambient temperature, then washed successively with water, sat. NaHCO₃, and brine, dried over MgSO₄, filtered, and concentrated. The product is purified by chromatography on silica gel.

### EXAMPLE 23

### (2S,3S,4S)-3-(tert-butyldimethylsilyloxy)-4-(4-methoxybenzyloxy)-2-methylpentanal

A solution of N-methoxy N-methyl (2S,3S,4S)-3-(tert-butyldimethyl-silyloxy)-4-(4-methoxybenzyloxy)-2-methylpentanamide (42.6 g) in 150 mL of THF is cooled to −78° C. and treated with 1.0 M diisobutylaluminum hydride in hexanes (20 mL) over 15 minutes. After 10 minutes, the mixture is treated with 10 mL of methanol and partitioned between 200 mL each of ether and sat. Rochelle's salt. The organic phase is washed with brine, dried over MgSO₄, filtered, and concentrated. The product is purified by chromatography on silica gel.

### EXAMPLE 24

### (2Z,4R,5S,6S)-5-(tert-butyldimethylsilyloxy)-6-(4-methoxybenzylox)-2-iodo-4-methylhept-2-ene

A suspension of dry ethyl triphenylphosphonium iodide (68.7 g) in 600 mL of THF is treated with 2.5 M n-butyllithium in hexane (64 mL) over 30 minites. After an additional 10 minutes, the red solution is added via cannula to a −78° C. solution of iodine (41.7 g) in 1400 mL of THF at such a rate that the internal temperature remains below −70° C. The yellow slurry is warmed to −20° C., and 1.0 M sodium hexamethyldisilazide in THF (147 mL) is added over 30 minutes. After an additional 15 minutes, the orange solution is cooled to −33° C., and a solution of (2S,3S,4S)-3-(tert-butyldimethylsilyloxy)-4-(4-methoxybenzyloxy)-2-methylpentanal (35.2 g) in 200 mL of THF is added over 15 minutes. The mix is stirred for 45 minutes, then warmed to ambient temperature, quenched by addition of 20 mL of methanol, and concentrated. The residue is filtered through silica gel using ether, and the eluate is washed successively with sat. Na₂S₂O₃ and brine, dried over MgSO₄, filtered, and concentrated. The product is purified by chromatography on silica gel.

### EXAMPLE 25

### (2Z,4R,5S,6S)-5-(tert-butyldimethylsilyloxy)-6-hydroxy-2-iodo-4-methylhept-2-ene

A solution of (2Z,4R,5S,6S)-5-(tert-butyldimethylsilyloxy)-6-(4-methoxybenzyloxy)-2-iodo-4-methylhept-2-ene (6.5 g) in 125 mL of CH₂Cl₂ is treated with water (6 mL) and 2,3-dichloro-5,6-dicyano-1,4-benzoquinone (3.2 g) for 3 hours. Saturated NaHCO₃ is added (20 mL), and the phases are separated. The organic phase is dried over MgSO₄, filtered, and concentrated. The product is purified by chromatography on silica gel.

### EXAMPLE 26

### (3S,4R,5Z)-3-(tert-butyldimethylsilyloxy)-4-methyl-5-iodo-hept-5-en-2-one

A solution of DMSO (7.2 mL) in 150 mL of CH₂Cl₂ is cooled to −78° C. and treated with oxalyl chloride (4.3 mL) over 30 minutes. After an additional 30 minutes, a solution of (2Z,4R,5S,6S)-5-(tert-butyldimethylsilyloxy)-6-hydroxy-2-iodo-4-methylhept-2-ene (11.9 g) in 10 mL of CH₂Cl₂ is added dropwise over 30 minutes. After an additional 45 minutes, diisopropylethylamine (34.5 mL) is added over 45 minutes. The mix is stirred for 30 minutes, then is allowed to warm to ambient temperature and poured into 200 mL of vigorously stirred 1.0 M NaHSO₄. The phases are separated, and the aqueous phase is extracted with ether. The extract is combined with the organic phase and concentrated. The residue is dissolved in ether and washed sequentially with aq. NaHSO₄, water, sat. NaHCO₃, and brine, then dried over MgSO₄, filtered, and evaporated to provide the product ketone.

### EXAMPLE 27

### (4R,5S,2Z,6E)-5-(tert-butyldimethylsilyloxy)-4,6-dimethyl-2-iodo-7-(2-methythiazol-4-yl)-hepta-2,6-diene

A 1.0 M solution of sodium hexamethyldisilazide in tetrahydrofuran (18 mL) is added dropwise to a −78° C. solution of (2-methythiazol-4-yl)methyl diphenylphosphine oxide (6.90 g) and (3S,4R,5Z)-3-(tert-butyldimethylsilyloxy)-4-methyl-5-iodo-hept-5-en-2-one (5.65 g) in 15 mL of THF. The mix is allowed to warm to ambient temperature, stirred for 10 hours, then is poured into sat. NH₄Cl and extracted with ether. The extract is washed sequentially with sat. NaHCO₃ and brine. The solution is dried over MgSO₄, filtered, and evaporated. The product is purified by silica gel chromatography.

### EXAMPLE 28

### (3Z,5R,6S,7E)-6-(tert-butyldimethylsilyloxy)-8-(2-methylthiazol-4-yl)-3,5,7-trimethyl-octa-1,3,7-triene

A mixture of tetrakis(triphenylphosphine)palladium (1.18 g) and lithium chloride (12.9 g) in 500 mL of THF is stirred under argon for 15 minutes, then a solution of (4R,5S,2Z,6E)-5-(tert-butyldimethylsilyloxy)-4,6-dimethyl-2-iodo-7-(2-methythiazol-4-yl)-hepta-2,6-diene (46.7 g) and vinyltributylstannane (31.6 g) in 250 mL of THF is added followed by an additional 25 mL of THF. The resulting solution is heated at reflux for 48 hours, then cooled and partitioned between 500 mL water and 250 mL of pentane. The aqueous phase is extracted with pentane, and the the extract is combined with the original organic phase and washed sequentially with sat. NaHCO₃ and brine. The solution is dried over MgSO₄, filtered, and evaporated. The product is purified by silica gel chromatography.

### EXAMPLE 29

### (3Z,5R,6S,7E)-6-hydroxy-8-(2-methylthiazol-4-yl)-3,5,7-trimethyl-octa-1,3,7-triene

A solution of (3Z,5R,6S,7E)-6-(tert-butyldimethylsilyloxy)-8-(2-methylthiazol-4-yl)-3,5,7-trimethyl-octa-1,3,7-triene (3.8 g) in 100 mL of acetonitrile is cooled on ice and treated dropwise with 5 mL of 48% hydrofluoric acid. After stirring for 1 hour, the mixture is quenched by careful addition of sat. NaHCO₃ and extracted with ethyl acetate. The extract is washed with brine, dried over MgSO₄, filtered, and evaporated. The product is purified by silica gel chromatography.

### EXAMPLE 30

### (14R)-10,11-dehydro-14-methyl-epothilone D

(14R)-10,11-dehydro-14-methyl-epothilone D is prepared according to the methods of the above examples 12-14 but using (3Z,5R,6S,7E)-6-hydroxy-8-(2-methylthiazol-4-yl)-3,5,7-trimethyl-octa-1,3,7-triene in place of (3Z,5S,6S,7E)-6-hydroxy-8-(2-methylthiazol-4-yl)-3,5,7-trimethyl-octa-1,3,7-triene in Example 12.

### EXAMPLE 31

### (14R)-14-methyl-epothilone D

A mixture of (14R)-10,11-dehydro-14-methyl-epothilone D (500 mg), dipotassium azodicarboxylate (500 mg), and acetic acid (0.5 mL) in 10 mL of anhydrous dioxane is stirred on ice for 1 hour, then is poured into sat. NaHCO₃ and extracted with ethyl acetate. The extract is washed with brine, then dried over MgSO₄, filtered, and evaporated. The product is purified by flash chromatography on SiO₂ (1:2 hexanes/ethyl acetate).

### EXAMPLE 32

### (14S)-14-methyl-epothilone B

A solution of freshly prepared 3,3-dimethyldioxirane (0.087 M in acetone, 3.6 mL) is added dropwise to a solution of (14R)-14-methyl-epothilone D (90 mg) in 1.8 mL of CH₂Cl₂ at −78° C. The solution was warmed to −50° C., kept for 1 hour, and an additional 1.0 mL of the 3,3-dimethyldioxirane is added. After stirring for an additional 1.5 hour, the solution was dried by passing a stream of nitrogen gas through the solution at −50° C. The residue is purified by silica gel chromatography.

### EXAMPLE 33

### (14R)-14-methyl-epothilone B

A solution of freshly prepared 3,3-dimethyldioxirane (0.087 M in acetone, 3.6 mL) is added dropwise to a solution of (14S)-14-methyl-epothilone D (90 mg) in 1.8 mL of CH₂Cl₂ at −78° C. The solution was warmed to −50° C., kept for 1 hour, and an additional 1.0 mL of the 3,3-dimethyldioxirane is added. After stirring for an additional 1.5 hour, the solution was dried by passing a stream of nitrogen gas through the solution at −50° C. The residue is purified by silica gel chromatography.

### EXAMPLE 34

### 4-benzyl-3-[3-hydroxy-2,4-dimethyl-5-(2-methyl-thiazol-4-yl)-pent-4-enoyl]-oxazolidin-2-one

A solution of 2-methyl-3-(2-methyl-thiazol-4-yl)-propenal (600 mg, 3.59 mmol), N-propionyl-(4S)-4-benzyl-2-oxazolidinone (700 mg, 3.00 mmol), magnesium bromide diethyl etherate (155 mg, 0.60 mmol), triethyl amine (0.836 mL, 6.00 mmol), trimethylsilyl chloride (0.571 mL, 4.50 mmol) and ethyl acetate (6 mL) was stirred at room temperature overnight. The reaction mixture was filtered through silica gel, which was then washed with ethyl acetate. The ethyl acetate was concentrated. The residue was dissolved in Methanol (50 mL) and trifluoroacetic acid (˜20 drops) was added. This was stirred for 20 min. The solution was concentrated and purified on silica gel (25% ethyl acetate/hexanes to 50% ethyl acetate/hexanes). This produced 4-benzyl-3-[3-hydroxy-2,4-dimethyl-5-(2-methyl-thiazol-4-yl)-pent-4-enoyl]-oxazolidin-2-one (1.18 g, 2.95 mmol, 98%). ¹H NMR (400 MHz, CDCl₃) δ 7.28 (m, 5 H), 6.99 (s, 1 H), 6.58 (s, 1 H), 4.71 (m, 1 H), 4.35 (d, 2 H), 4.20 (m, 1 H), 3.32 (d, 1 H), 2.78 (d, 2 H), 2.72 (s, 3 H), 2.13 (s, 3 H), 1.15 (d, 3 H); ¹³C NMR δ 176.4, 164.6, 153.7, 152.4, 139.1, 135.3, 129.4, 128.8, 127.2, 121.8, 116.1, 81.3, 65.9, 55.5, 40.6, 37.7, 19.0, 14.7, 13.4.

### EXAMPLE 35

### 4-Benzyl-3-[2,4-dimethyl-5-(2-methyl-thiazol-4-yl)-3-triethylsilanyloxy-pent-4-enoyl]-oxazolidin-2-one

A solution of 4-benzyl-3-[3-hydroxy-2,4-dimethyl-5-(2-methyl-thiazol-4-yl)-pent-4-enoyl]-oxazolidin-2-one (920 mg, 2.30 mmol) and imidazole (235 mg, 3.40 mmol) in DMF (5 mL) was cooled to 0° C. Added to this was chlorotriethyl silane (0.463 mL, 2.80 mmol). This was allowed to warm to room temperature and stirred for 3 hours. The mixture was diluted with ethyl acetate and quenched with water. The layers were separated and the organic solution was washed with brine, dried over MgSO₄, filtered and concentrated. The crude material was purified on silica gel (10% ethyl acetate/hexanes). To provide 4-benzyl-3-[2,4-dimethyl-5-(2-methyl-thiazol-4-yl)-3-triethylsilanyloxy-pent-4-enoyl]-oxazolidin-2-one (871 mg, 1.69 mmol, 74%). ¹H NMR (400 MHz, CDCl₃) δ 7.31 (m, 5 H), 7.02 (s, 1 H), 6.56 (s, 1 H), 4.7 (m, 1 H), 4.52 (d, 2 H), 4.14 (m, 1 H), 3.41 (d, 1 H), 2.71 (s, 3 H), 1.56 (s, 3 H), 1.00 (d, 3 H), 0.92 (t, 9 H), 0.57 (q, 6 H); ¹³C NMR δ 175.9, 164.4, 153.1, 152.5, 139.4, 135.6, 129.4, 128.9, 127.2, 122.4, 115.7, 81.9, 65.7, 55.4, 42.1, 38.1, 19.1, 14.6, 13.3, 6.8, 4.8.

### EXAMPLE 36

### 2,4-Dimethyl-5-(2-methyl-thiazol-4-yl)-3-triethylsilanyloxy-pent-4-en-1-ol

A solution of 4-benzyl-3-[2,4-dimethyl-5-(2-methyl-thiazol-4-yl)-3-triethylsilanyloxy-pent-4-enoyl]-oxazolidin-2-one (871 mg, 1.69 mmol) in THF (79.2 mL) was cooled to 0° C. Added to this was methanol (0.317 mL), followed by LiBH₄ (193 mg, 8.86 mmol). This was stirred at 0° C. for 1 hour, then at room temperature overnight. The reaction was quenched by careful addition of ¹N NaOH (8 mL). The layers were separated and the organic solution was extracted with brine, dried over MgSO₄, filtered, and evaporated. The crude material was purified on silica gel (50% ethyl acetate/hexane) to provide 2,4-dimethyl-5-(2-methyl-thiazol-4-yl)-3-triethylsilanyloxy-pent-4-en-1-ol (333 mg, 0.975 mmol, 58%). ¹H NMR (400 MHz, CDCl₃) δ 6.97 (s, 1 H), 6.47 (s, 1 H), 4.01 (d, 1 H), 3.66 (ap t, 2 H), 2.77 (s, 3H), 1.94 (m, 1 H), 1.59 (s, 3 H), 0.95 (t, 9 H), 0.81 (d, 3 H), 0.63 (q, 6 H); ¹³C NMR δ 164.5, 152.7, 140.2, 121.0, 115.6, 85.4, 67.3, 38.7, 19.2, 14.2, 14.0, 6.8, 4.7.

### EXAMPLE 37

### 4-(5-Iodo-2,4-dimethyl-3-triethylsilyloxy-pent-1-enyl)-2-methyl-thiazole

2,4-Dimethyl-5-(2-methyl-thiazol-4-yl)-3-triethylsilanyloxy-pent-4-en-1-ol (333 mg, 0.975 mmol) was dissolved in a solution of Et₂O/Acetonitrile (3:1, 5.72 mL) and cooled to 0° C. Added to this was imidazole (199 mg, 2.92 mmol), triphenyl phosphine (384 mg, 1.46 mmol), and iodine (371 mg, 1.46 mmol). The reaction mixture was stirred for 3 hours at 0° C. The reaction was carefully quenched with sat. Na₂S₂O₃, then diluted with ether (20 mL). The layers were separated and the organic layer was washed with brine, dried over MgSO₄, filtered and concentrated. The crude material was purified on silica gel (3% ethyl acetate/hexane) to provide 4-(5-Iodo-2,4-dimethyl-3-triethylsilyloxy-pent-1-enyl)-2-methyl-thiazole (300 mg, 0.664 mmol, 68%%). ¹H NMR (400 MHz, CDCl₃) δ 6.96 (s, 1 H), 6.46 (s, 1 H), 3.83 (d, 1 H), 3.48 (dd, 2 H), 2.71 (s, 3H), 1.53 (m, 1 H), 1.26 (s, 3 H), 0.94 (t, 9 H), 0.86 (d, 3 H), 0.62 (q, 6 H).

### EXAMPLE 38

### 4-(5-triphenylphosphonium-2,4-dimethyl-3-triethylsilyloxy-pent-1-enyl)-2-methyl-thiazole iodide

4-(5-Iodo-2,4-dimethyl-3-triethylsilanyloxy-pent-1-enyl)-2-methyl-thiazole (300 mg, 0.664 mmol) and triphenyl phosphine (190 mg, 0.724 mmol) were mixed together and heated neat to 100° C. After 1.5 hours the reaction was cooled to room temperature. The crude material was purified on silica gel (5% methanol/CH₂Cl₂) to produce (268 mg, 0.375 mmol, 56%). ¹H NMR (400 MHz, CDCl₃) δ 7.80 (m, 15 H), 6.97 (s, 1 H), 6.56 (s, 1 H), 4.12 (d, 1 H), 3.63 (m, 2 H), 2.70 (s, 3H), 2.17 (m, 1 H), 1.88 (s, 3 H), 0.95 (t, 9 H), 0.67 (ap q, 9 H); ¹³C NMR δ 164.7, 152.4, 138.3, 135.2, 133.8, 133.7, 130.7, 130.6, 122.16, 118.9, 118.1 116.7, 83.0, 32.9, 19.3, 18.0, 14.2, 7.0, 4.8.

### EXAMPLE 39

### 2,4-dimethyl-1-(2-methyl-thiazol-4-yl)-hexa-1,5-dien-3-ol

A solution of potassium tert-butoxide (1.93 g, 17.19 mmol) in THF (19 mL) was cooled to −78° C. Added to this via cannula was trans-2-butene (5.38 mL, 59.80 mmol) followed by n-butyl lithium (1.6 M in hexanes, 12.1 mL, 19.43 mmol). The mixture was allowed to warm to −45° C. for 30 minutes, then cooled back down to −78° C. Slowly added was a solution of (−)-B-methoxydiisopinocampheylborane (7.8 g, 24.67 mmol) in THF (78 mL). This was stirred at −78° C. for 1 hour. At this point, boron trifluoride diethyl etherate (3.5 mL, 27.66 mmol) was added the solution was stirred for 0.5 hour. A solution of 2-methyl-3-(2-methyl-thiazol-4-yl)-propenal (1.25 g, 7.47 mmol) in THF (12 mL) was added and this was stirred for 1.5 hours. Carefully added was 3 N NaOH (150 mL) and 30% H₂O₂ (150 mL). This was allowed to warm to room temperature and was stirred for 1 hour. The solution was extracted with ether (200 mL) and the organic layer was washed with brine (150 mL), dried over MgSO₄, filtered and concentrated. The crude material was purified on silica gel (10% ethyl acetate/hexane) to produce 2,4-dimethyl-1-(2-methyl-thiazol-4-yl)-hexa-1,5-dien-3-ol (1.24 g, 5.55 mmol, 74%). ¹H NMR (400 MHz, CDCl₃) δ 6.91 (s, 1 H), 6.48 (s, 1 H), 5.79 (m, 1 H), 5.11 (ap t, 2 H), 3.81 (d, 1 H), 2.66 (s, 3 H), 2.51 (br s, 1 H), 2.39 (m, 1 H), 1.98 (s, 3 H), 0.92 (d, 3 H); ¹³C NMR δ 164.5, 152.7, 140.8, 140.1, 121.1, 116.2, 115.5, 81.4, 42.0, 19.07, 16.8, 13.8.

### EXAMPLE 40

### 3,7-bis(tert-butyldimethylsilyl)epothilone D

Epothilone D (2.88 g, 5.9 mmol, 1 eq) was dissolved in dry CH₂Cl₂ (40 mL). The solution was chilled to −78° C. under N₂. Triethylamine (4.9 mL, 35.2 mmol, 6 eq) was added followed by tert-butyl-dimethylsilyl-triflate (5.5 mL, 23.5 mmol, 4 eq). The reaction was allowed to warm to −50° C. over the course of an hour while stirring under N₂. The remaining triflate was quenched by pouring the reaction into saturated NaHCO₃. The aqueous layer was extracted with CH₂Cl₂ (100 mL, 4×). The pooled organic portions were washed with brine (100 mL, 1×), dried over Na₂SO₄, filtered, and evaporated in vacuo to yield a yellow oil. The oil was applied to a silica flash column (6×4 cm) and eluted with 0, 10, and 20% ether in hexanes. Fractions eluting in 10-20% ether in hexanes were pooled and concentrated, yielding the product as a white foam (3.61 g, 5.0 mmol, 85%).

### EXAMPLE 41

### 3,7-bis(tert-butyldimethylsilyl)-12,13-dihydroxyepothilone D

Protected EpoD 2 (1.41 g, 1.96 mmol, 1 eq) and N,N,N′,N′-tetra-methylethylenediamine (310 mL, 2.06 mmol, 1.05 eq) were dissolved in dry CH₂Cl₂ (13 mL). The clear solution was brought to −78° C. under N₂. A solution of osmium tetroxide (535 mg, 2.06 mmol, 1.05 eq) in CH₂Cl₂ (7 mL) was then added. The resulting black solution was stirred for 45 minutes at −78° C. Aqueous saturated NaHSO₃ (25 mL) and THF (17 mL) were added and the suspension stirred at 65° C. for 12 hours. After stirring at room temperature for an additional 2 days, the reaction was concentrated in vacuo to a red-beige solid. Suspended the solid in CH₂Cl₂ and filtered through celite. The celite was rinsed copiously with CH₂Cl₂. The filtrate was washed with aqueous saturated NaHCO₃ (1×) and brine (1×), dried over Na₂SO₄, filtered, and concentrated in vacuo to a brown oil. The crude material was purified on a 110 g silica column eluting with 0, 10, 20, 30, 40, 50, 60, and 75% Eethyl acetate in hexanes. The product eluted in the 40% ethyl acetate/hexanes fractions and was concentrated in vacuo to yield the product as a white foam (1.11 g, 1.47 mmol, 75%). R_{f}: 0.4 (silica gel, 30% EtOAc/Hex). LRMS: (M+H) 754.47.

### EXAMPLE 42

### Compound (6)

Step 1. Ketoacid: 3,7-bis(tert-butyldimethylsilyl)-12,13-dihydroxy-epothilone D (1.07 g, 1.42 mmol, 1 eq) was dissolved in benzene (14 mL). Pb(OAc)₄ (693 mg, 1.56 mmol, 1.1 eq) was added and the reaction stirred at room temperature under N₂ for 30 minutes. To the resulting cloudy yellow solution was added K₂CO₃ (1.96 g, 14.2 mmol, 10 eq) and MeOH (14 mL). The clear solution was brought to 65° C. and stirred for 2 hours. After concentrating in vacuo, the reaction was partitioned between H₂O (pH=2 adjusted with 2N HCl) and Et₂O. The acidic aqueous layer was extracted with Et₂O (4×). The pooled organic layers were washed with brine (1×), dried over Na₂SO₄, filtered and concentrated in vacuo to an orangish-yellow oil. The crude ketoacid was used without further purification in the subsequent esterification. R_{f}: 0.5 (silica gel, 30% EtOAc/Hex). LRMS: (M+Na) 581.38.

Step 2. Esterification: The crude ketoacid from Step (approximately 794 mg, 1.42 mmol, 1 eq) was dissolved in methanol (4.4 mL) and toluene (15 mL). (Trimethylsilyl)diazomethane (2.8 mL of a 2M solution in Et₂O, 5.68 mmol, 4 eq) was added dropwise to the clear red solution resulting in substantial gas evolution. The reaction was stirred at room temperature under N₂ for 2 hours. The reaction was concentrated in vacuo and purified on a 35 g silica column eluting with 0, 5, 10, 15, 20, and 30% ethyl acetate in hexanes. The product eluted in the 15-20% ethyl acetate/hexanes fractions and was concentrated in vacuo to an yellow oil (0.6618 g, 1.16 mmol, 81% over 2 steps). R_{f}: 0.4 (silica gel, 15% EtOAc/Hex). LRMS: (M+H) 574.0.

Step 3. The keto-ester from Step 2 (77.8 mg, 0.14 mmol, 1 eq) was dissolved in dry toluene (1 mL). A solution of dimethyltitanocene in toluene (1 mL, approximately 14% w/w as determined by ¹H NMR) was added. The resulting orange solution was brought to 70° C. under N₂ and stirred for 1 hour, after which point heating was discontinued and the reaction stirred at room temperature for an additional 12 hours. Hexane (10 mL) was added to precipitate the titanocene complex. The precipitate was filtered through a plug of celite and washed with ether. The filtrate was concentrated in vacuo to yield an orange residue. The residue was purified on a silica flash column (2×10 cm) and eluted with 0, 2.5, 5, and 10% ether in hexanes. Fractions eluting in 5% ether/hexanes were pooled and concentrated in vacuo as a colorless, opaque oil to provide the alkenyl-ester (59.9 mg, 0.10 mmol, 74%).

R_{f}: 0.4 (silica gel, 10% EtOAc/Hex). LRMS: (M+H) 571.0.

Step 4. Ester hydrolysis. The alkenyl-ester from Step 3 (70.9 mg, 0.12 mmol, 1 eq) was dissolved in i-propanol (2 mL). LiOH (500 mL of 5 M solution in H₂O, 2.48 mmol, 20 eq) and H₂O (500 mL) were added resulting in a homogeneous solution. The reaction was stirred at room temperature for 48 hours. The solution was acidified to pH 2 with 2 N HCl. The reaction was partitioned between CH₂Cl₂ (10 mL) and H₂O (10 mL) and extracted with CH₂Cl₂ (4×). The pooled organics were washed with brine (1×), dried over Na₂SO₄, filtered, and concentrated in vacuo to an yellow oil. The crude material was applied to a silica flash column (0.5×8 cm) and eluted with 0, 5, 10, and 15% EtOAc/Hex. Fractions eluting in 5-10% EtOAc/Hex were pooled and concentrated, yielding acid compound (6) as an opaque oil (59.6 mg, 0.11 mmol, 86%). R_{f}: 0.6 (silica gel, 25% EtOAc/Hex). LRMS: (M+H) 557.0.

### EXAMPLE 43

### (14S)-14-methylepothilone D

Step 1. Compound (6) (Example 42) (320 mg, 0.575 mmol, 1 eq), 2,4-dimethyl-1-(2-methyl-thiazol-4-yl)-hexa-1,5-dien-3-ol (Example 39) (210 mg, 0.834 mmol, 1.45 eq), and 4-dimethylamino-pyridine (35 mg, 0.288 mmol, 0.5 eq) were dissolved in CH₂Cl₂ (3 mL). The solution was brought to 0° C. under N₂. 1-[3-(dimethylamino)propyl]-3-ethylcarbodiimide hydrochloride (220 mg, 1.15 mmol, 2 eq) was added, and the reaction was stirred at 0° C. for 30 minutes. The reaction was warmed to room temperature and stirred for 4 hours. The reaction was concentrated in vacuo to an oil. The oil was partitioned between ethyl acetate (25 mL) and saturated aq. NH₄Cl (25 mL). The organic layer was washed with saturated aq. NH₄Cl (1×) and brine (1×), dried over Na₂SO₄, filtered, and concentrated in vacuo. The oil was applied to a silica flash column (2×8 cm) and eluted with 0, 5, 10, 15, 20, 30, and 40% ethyl acetate/hexanes. Fractions eluting in 10% ethyl acetate/hexanes were pooled and concentrated in vacuo to yield the diene product as a colorless, opaque oil (349 mg, 0.46 mmol, 80%). R_{f}: 0.25 (silica gel, 10% Et₂O/Hex). LRMS: (M+H) 763.0.

Step 2. The diene from Step 1 (38 mg, 0.052 mmol, 1 eq) was dissolved in dry toluene (21 mL, 2.5 mM). The clear, colorless solution was brought to 110° C. under N₂. Tricyclohexylphosphine-[1,3-bis(2,4,6-trimethylphenyl)-4,5-dihydroimidazol-2-ylidene][benzylidene]-ruthenium(IV)dichloride (10 mg) was added and the initially red solution quickly became greenish black. The reaction was stirred at 110° C. for 5 days with regular additions of the ruthenium catalyst (20 mg, 10 mg, and 35 mg). Methylsulfoxide (5 mL) was added to aid removal of the ruthenium. The reaction was stirred for 12 hours at room temperature. The black solution was applied directly to a plug of silica (6×3 cm) and eluted with 50% ethyl acetate/hexanes (100 mL). The filtrate was concentrated in vacuo to a black oil. The crude material was applied to a silica flash column (0.5×6 cm) and eluted with 0, 5, 7.5, 10, 12.5, and 20% ether/hexanes. Fractions eluting in 10-12.5% ether/hexanes were pooled and concentrated in vacuo, yielding a colorless, opaque oil (12 mg of an approximately 1:1 mixture of cis- and trans-isomers). R_{f}: 0.45 (silica gel, 10% EtOAc/Hex). LRMS: 2 peaks with the same weight (M+H) 735.0.

Step 3. The product from Step 2 (10 mg, 0.014 mmol, 1 eq, an approximately 1:1 mixture of cis- and trans-isomers ) was dissolved in dry CH₂Cl₂ (300 mL). The clear solution was brought to 0° C. under N₂. Trifluoroacetic acid (200 mL) was added, and the reaction was warmed to room temperature over 1 hour. The reaction was quenched by addition of saturated NaH₂CO₃, and the aqueous layer was extracted with CH₂Cl₂ (3×). The pooled organic layers were washed with brine (1×), dried over Na₂SO₄, filtered, and concentrated in vacuo to an yellow oil. The crude material was applied to silica Pasteur pipette column and eluted with 0, 10, 20, 25, 30, and 35% ethyl acetate/hexanes. Fractions eluting in 25-30% ethyl acetate/hexanes were pooled and concentrated in vacuo to yield a mixture of the cis- and trans-isomers. The mixture was applied to a second Pasteur pipette column and eluted with 20, 30, 40, 50, and 60% tert-butyl-methyl ether/hexanes. Fractions eluting in the 50% tert-butyl-methyl ether/hexanes fractions provided the pure 12,13-cis-isomer (0.6 mg, 0.001 mmol, 2% over 2 steps). R_{f}: 0.4 (silica gel, 60% tert-butyl-methyl ether/Hex). HRMS: (M+H) 506.2947.

The invention having now been described by way of written description, those of skill in the art will recognize that the invention can be practiced in a variety of embodiments, and that the foregoing description and examples, while describing the best mode contemplated by the inventors, is for purposes of illustration and not limitation of the following claims. All references cited herein, including patents, patent applications, PCT publications, papers, text books, and the like, and the references cited therein, to the extent that they are not already, are hereby incorporated herein by reference in their entirety.

What is claimed is:

1. A compound of the structure 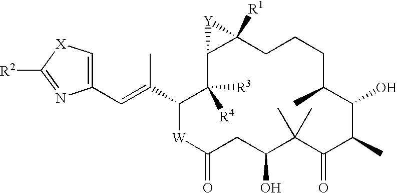
wherein R¹ is H or C₁-C₄ alkyl;
R² is C₁-C₃ alkyl, CH₂OH, CH₂NH₂, or CH₂F;
R³ is Me;
R⁴ is H;
W is O or NH;
X is S or O; and
Y is O or a bond.

2. A compound of claim 1 wherein R¹ is Me.

3. A compound of claim 2 wherein Y is a bond.

4. A compound of claim 3 wherein X is S.

5. A compound of claim 3 wherein X is O.

6. A compound of claim 4 selected from the group consisting of

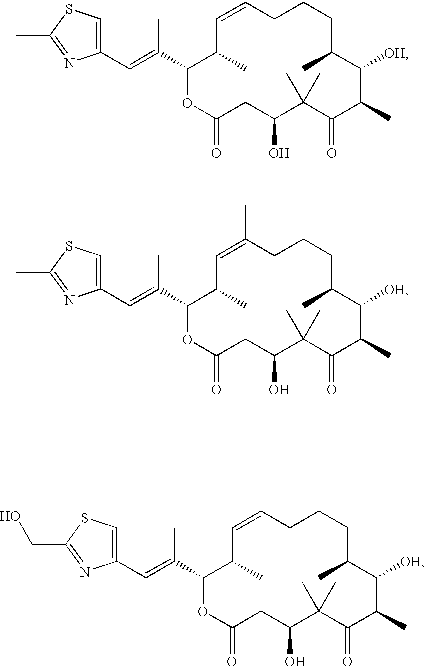

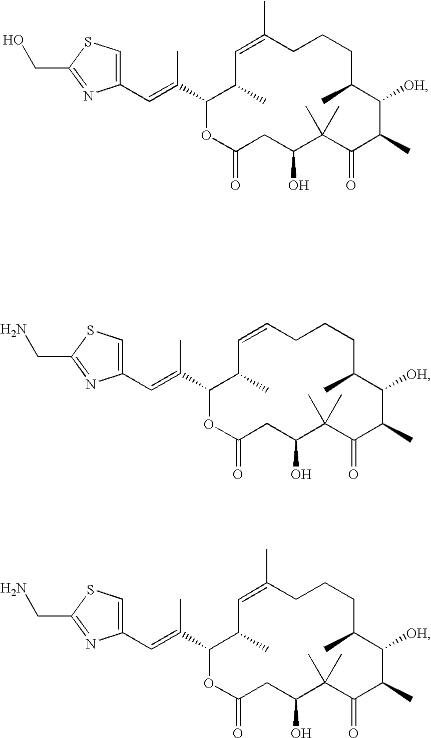

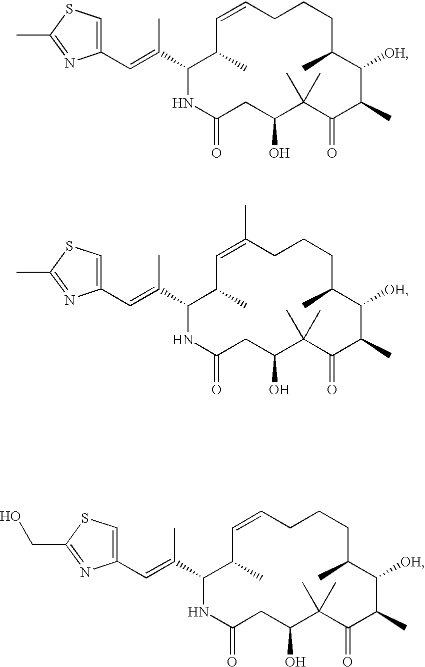

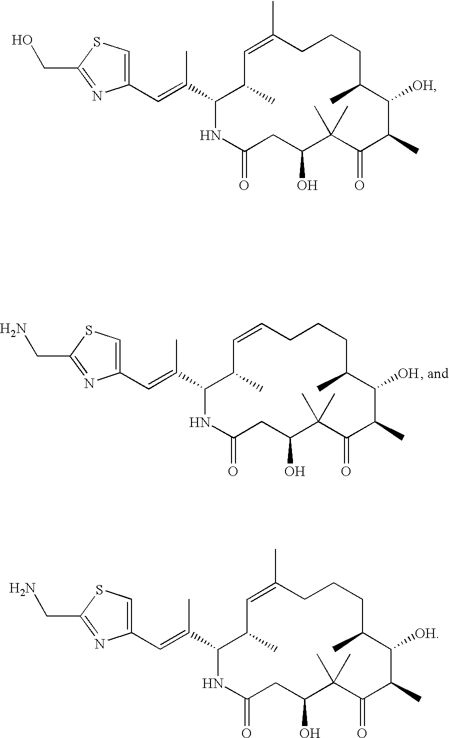

7. A compound of Claim 5 selected from the group consisting of

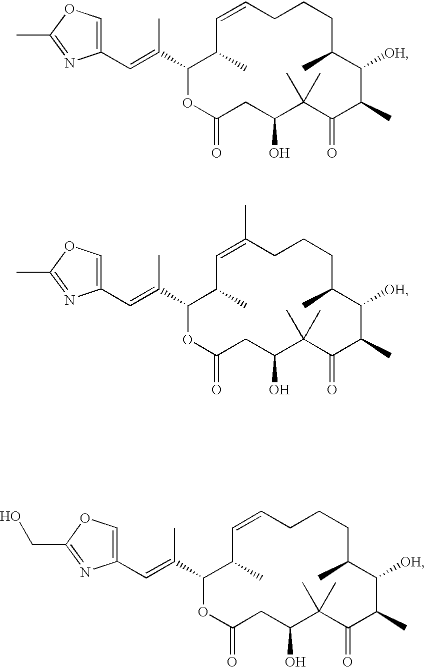

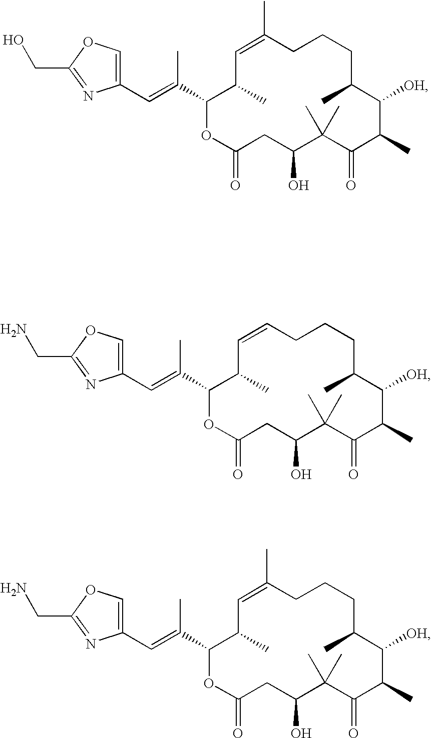

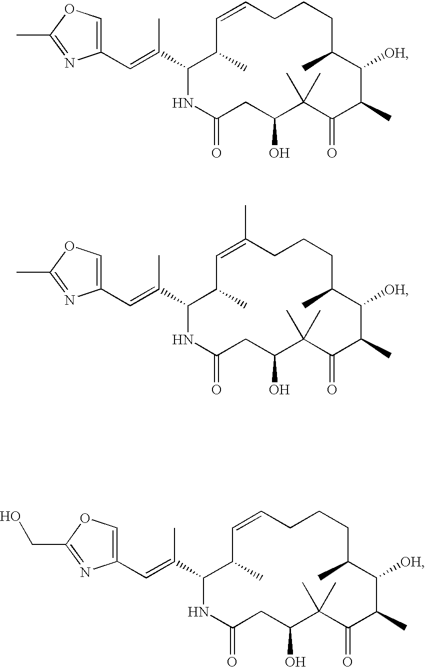

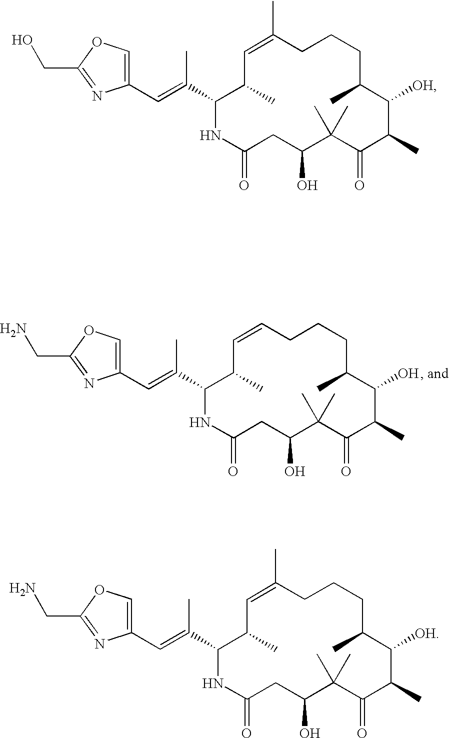

8. A compound of claim 1 selected from the group consisting of

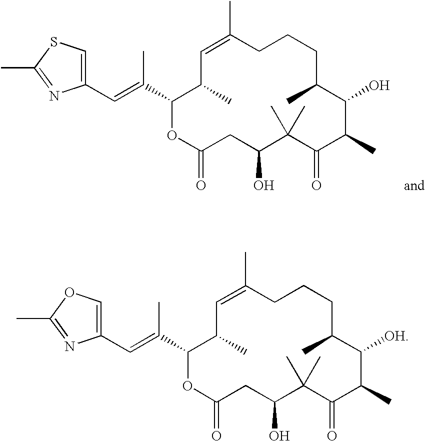

9. A compound of claim 1 selected from the group consisting of

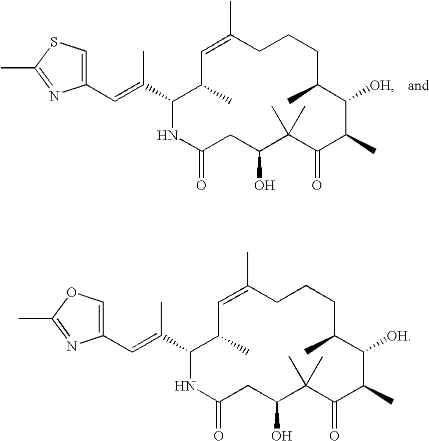

10. A compound of claim 1 wherein Y is O.

11. A compound of claim 10 wherein X is S.

12. A compound of claim 10 wherein X is O.

13. A compound of claim 11 selected from the group consisting of

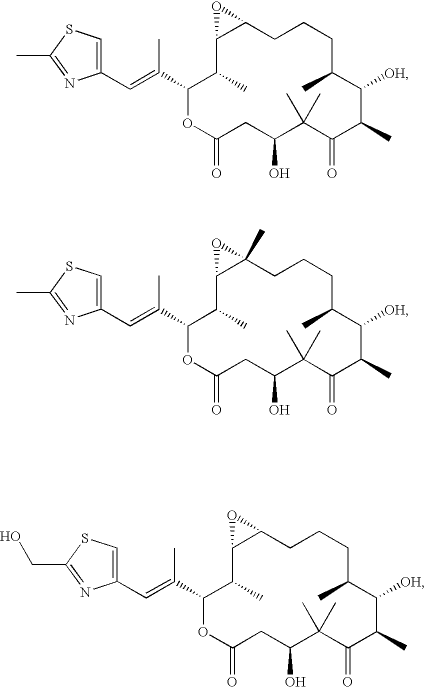

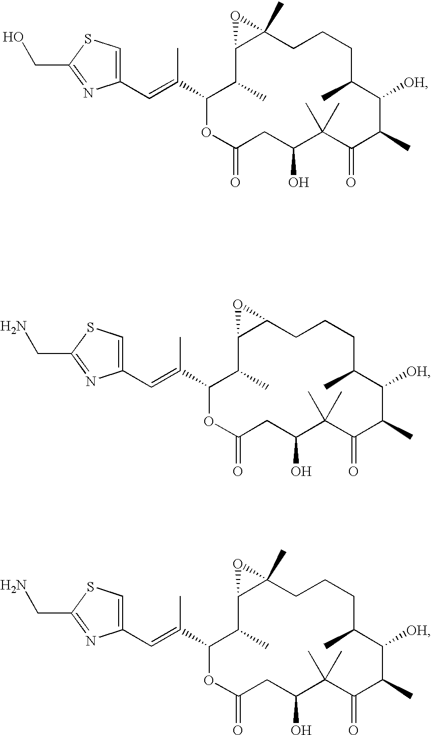

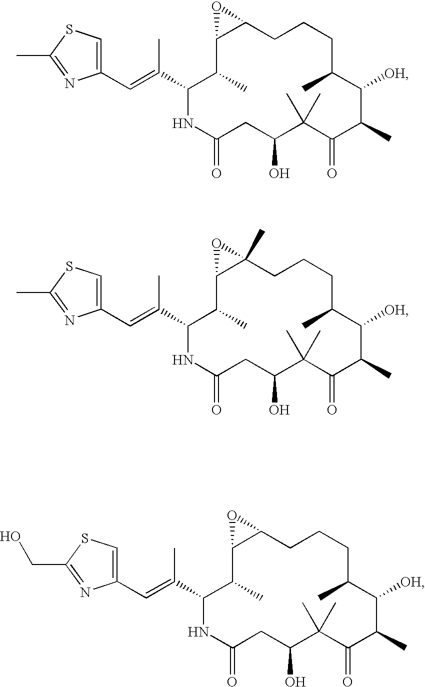

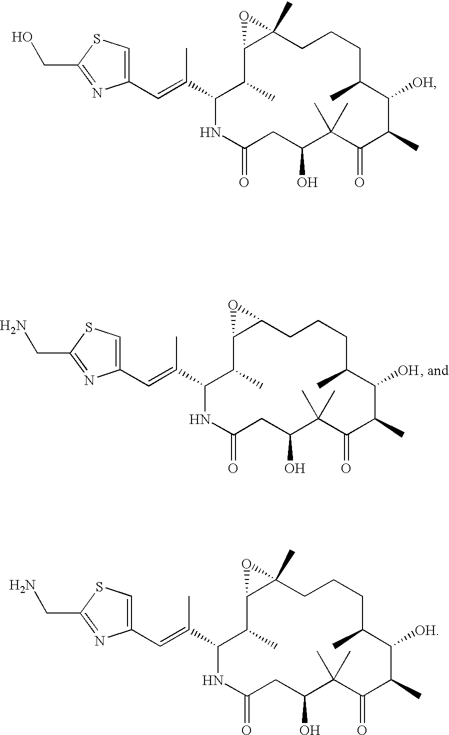

14. A compound of claim 12 selected from the group consisting of

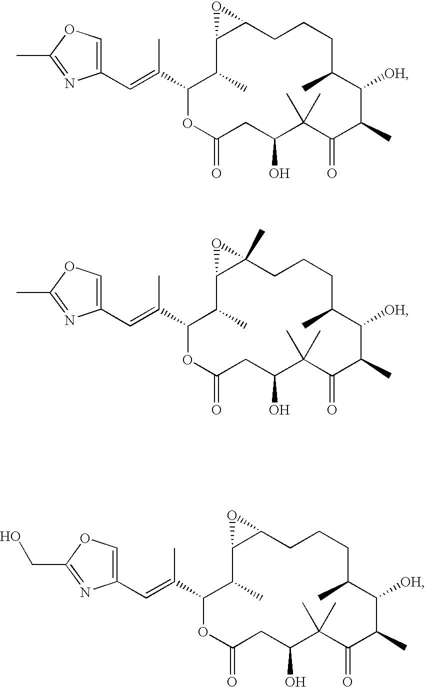

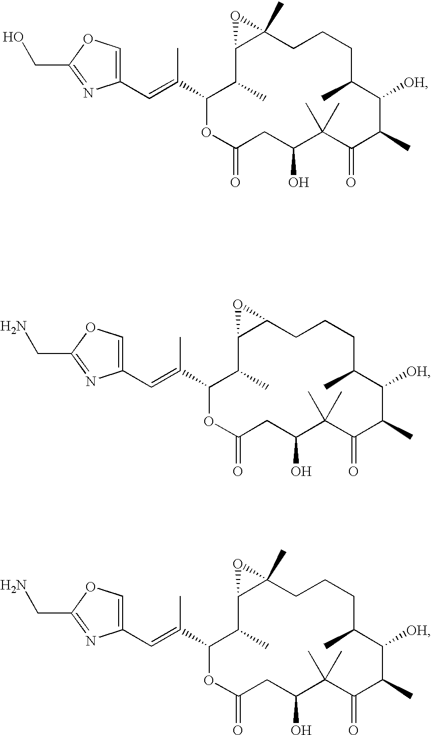

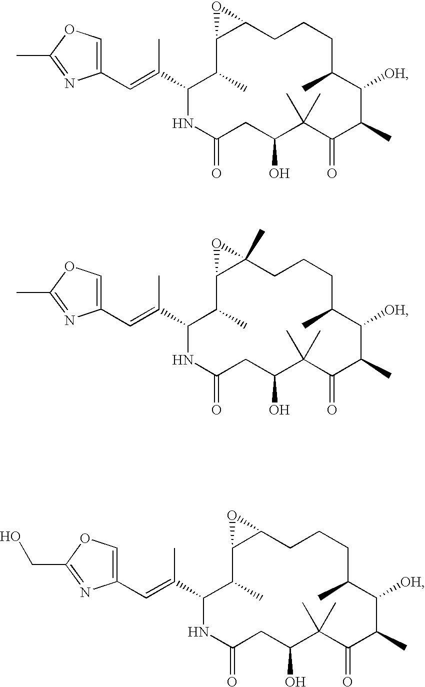

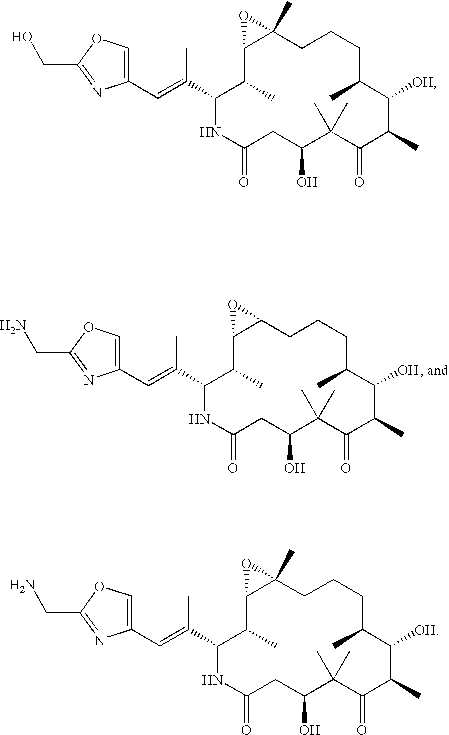

15. A compound of claim 1 selected from the group consisting of

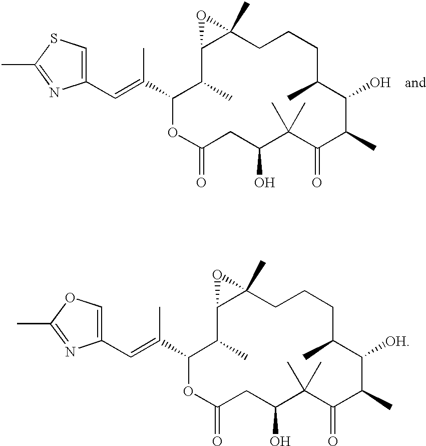

16. A compound of claim 1 selected from the group consisting of

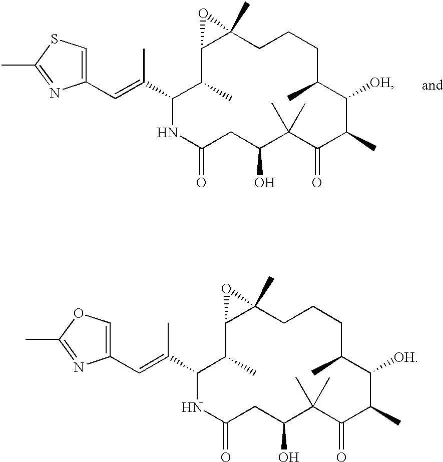

17. A composition comprising a compound of claim 1 together with a pharmaceutically acceptable carrier.

18. The composition of claim 17 wherein the compound of claim 1 is selected from the group consisting of

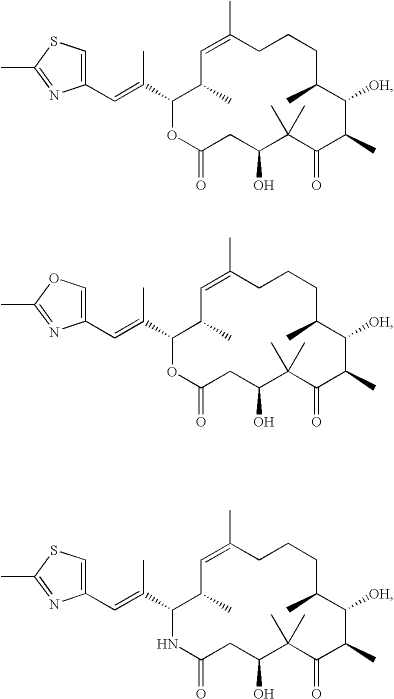

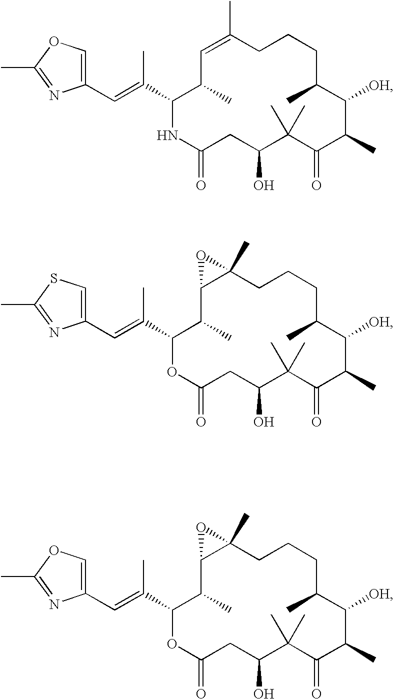

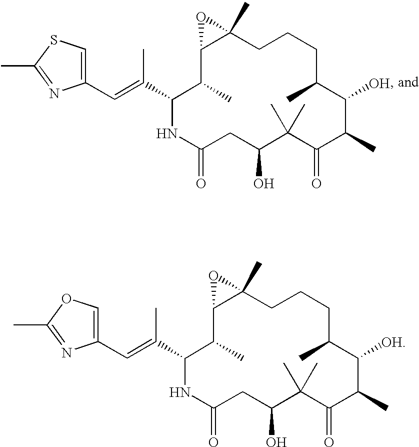

19. A method for treatment of a disease or condition characterized by undesired cellular hyperproliferation comprising administering to a subject in need of treatment a therapeutically effective dose of a composition of claim 19.

20. The method of claim 19 wherein the disease or condition characterized by undesired cellular hyperproliferation is cancer.
